(19) United States
(12) Patent Application Publication
(10) Pub. No.: US 2005/0112737 A1
(43) Pub. Date: May 26, 2005

(54) Lactic acid producing yeast

(75) Inventors: Chi Li Liu, Decatur, IL (US); Jefferson C. Lievense, Forsyth, IL (US)

Correspondence Address: WILLIAMS, MORGAN & AMERSON, P.C., 10333 RICHMOND, SUITE 1100, HOUSTON, TX, 77042, US

(73) Assignee: A. E. Staley Manufacturing Co.

(21) Appl. No.: 10/717,993

(22) Filed: Nov. 20, 2003

(51) Int. Cl. C12P 7/56; C12N 1/18

(52) U.S. Cl. 435/139; 435/254.2

(57) ABSTRACT

Disclosed herein are yeasts, which, when cultured, can produce relatively high concentrations of lactic acid. Also disclosed herein are culture media that result in relatively lower levels of by-product impurities when lactic acid-producing yeast are cultured in them.

### BACKGROUND OF THE INVENTION

The present invention relates generally to yeasts (e.g., fungi), which, when cultured, can produce relatively high concentrations of lactic acid. The present invention also relates to a culture medium that results in relatively lower levels of by-product impurities when lactic acid-producing yeast are cultured in it than when the yeast are cultured in certain media known in the art.

Lactic acid (2-hydroxypropionic acid, CH₃CHOHCOOH) is a naturally occurring hydroxyl acid that can be produced by fermentation or chemical synthesis. Lactic acid is the simplest hydroxyl acid that is optically active. L(+)-lactic acid can be produced directly without D(−)-lactic acid through fermentation (e.g., known chemical syntheses produce racemic mixtures of both isomers). Likewise D(−)-lactic acid can be produced by fermentation without L(+)-lactic acid. Lactic acid can be used in food as a preservative and flavor enhancer. Lactic acid derivatives can be used in industrial applications, such as paint and electrodeposition coating, pharmaceuticals and cosmetics. An important compound that can be produced by the dehydration of lactic acid is poly(lactic acid) plastic. L(+)-lactic acid is the preferred polymerization feedstock for biodegradable plastic applications.

L(+)-lactic acid fermentation can be carried out by cultivating certain microorganisms, such as certain Lactobacillus, Bacillus, Lactococcus, or Rhizopus, in a batch process. One of the problems that can be encountered in lactic acid fermentation is the inhibition of growth and metabolism caused by the accumulation of the undissociated acid (e.g., decreasing the pH of the fermentation broth) (Buchta, 1983; Hongo et al., 1985; Benninga, 1990). In certain cases, the pH of the fermentation process can be controlled to be at or near neutral by the addition of reagents such as Ca(OH)₂, CaCO₃, NaOH, or NH₄OH to the fermentation. As a consequence, the resulting fermentation broth can contain high concentrations of various salts, and recovery of undissociated lactic acid from the broth can be costly. Furthermore, certain Lactobacillus can be fastidious in that they can require a complex nitrogen source, such as yeast extract or corn steep liquor (CSL), for the production of lactic acid (WO 99/19503). Such complex nitrogen sources can comprise additional organic and inorganic impurities that can complicate recovery of lactic acid.

Another method of relieving the inhibition caused by the accumulation of lactic acid in the culture medium, involves the continuous removal of lactic acid from a fermentation broth during fermentation of, for example, certain Rhizopus species. A resin, such as polyvinylpyridine, can be used for such continuous removal.

Preferably lactic acid is recovered and purified to the highest possible level of purity when used as a polymer grade feedstock. The organic impurities (derived from complex nitrogen sources, for example), the inorganic impurities (related to media ingredients and neutralizing agents), and the metabolic intermediates secreted by the production organism during the fermentation are preferably all removed.

Wild type S. cerevisiae transformed with a lactate dehydrogenase-bearing (e.g., LDH-bearing) plasmid can produce some lactic acid when cultured. However, the concomitant ethanol formation from glucose by the recombinant yeast cells can result in three complications. First, the glucose used for ethanol production during the lactic acid fermentation is a carbon loss, which reduces the yield of lactic acid when calculated on a per gram glucose basis. Second, accumulation of ethanol in the broth lowers the fermentation efficiency for lactic acid production. And third, ethanol actually is an impurity, which needs to be removed during the purification process for lactic acid.

In certain yeasts, notably Crabtree positive yeasts, the main pathway for decarboxylation of pyruvate involves pyruvate decarboxylase. Crabtree positiveyeasts produce alcohol from pyruvate in the presence of excess sugar (e.g., glucose) under aerobic conditions or when the growth rate of the culture is higher than the critical growth rate. Examples of Crabtree positive yeasts are Saccharomyces cerevisiae, Candida glabrata, and Schizosaccharomyces pombe. In order to redirect carbon flux away from ethanol production to improve the yield of lactic acid, which is derived from the pyruvate, it is desirable to limit the decarboxylation of pyruvate in yeasts.

In S. cerevisiae pyruvate decarboxylase (EC 4.1.1.1) catalyzes the conversion of pyruvate to acetaldehyde, and this is the first step in fermentative metabolism. When pyruvate decarboxylase structural genes are disrupted in S. cerevisiae the yeast cannot produce ethanol (Hohmann, 1997). It has been proposed that in addition to catabolic activity, pyruvate decarboxylase also serves a biosynthetic function. It is known in the art that pyruvate decarboxylase-negative (Pdc⁻) (e.g., having no detectable amount of pyruvate decarboxylase activity) Saccharomyces cerevisiae strains cannot grow on synthetic culture medium in an aerobic glucose-limited chemostat, when glucose is the sole carbon source, without the addition of small amounts of ethanol or acetate (e.g., 5% of carbon required for growth).

### SUMMARY OF THE INVENTION

Certain embodiments of the present invention are directed to acid-tolerant (AT) yeast strains. The AT yeast strains produce essentially no ethanol when cultured in a culture medium, and they comprise a genome that includes an exogenous lactate dehydrogenase (LDH) gene. Preferably the exogenous LDH gene is a L-lactate dehydrogenase gene. The exogenous LDH gene can be an element of at least one chromosome of an AT yeast and/or at least one plasmid present in the AT yeast can comprise the exogenous LDH gene. The LDH can be expressed in the AT yeast strain, and its expression results in a lactate dehydrogenase protein having lactate dehydrogenase activity. In some embodiments, the AT yeast strain has no detectable amount of pyruvate decarboxylase activity. In certain embodiments, a wild type strain of the AT yeast strain is Crabtree positive. Furthermore, an AT yeast strain is capable of producing lactic acid in a minimal medium at a lower pH than its parent yeast strain. A parent strain of an AT yeast strain also produces essentially no ethanol when cultured in a culture medium and, has a genome that includes an exogenous lactate dehydrogenase (LDH) gene that can be expressed, such that the resulting protein has lactate dehydrogenase activity. However, the parent strain has not undergone manipulation (e.g., selection) that results in its being acid tolerant.

It is preferred that an AT yeast strain is capable of producing lactic acid at a pH of less than about 3.5, more preferably a pH less than about 2.8, and most preferably at a pH of less than about 2.3. It is also preferred that the AT yeast strain is capable of producing greater than about 500 mM lactic acid in a culture broth, when cultured aerobically in a minimal medium (e.g., in batch culture, in fed-batch culture, or chemostat). Preferably the AT yeast strain is capable of producing 500 mM lactic acid in culture broth at a pH between about 2.3 and 2.4. More preferably the AT yeast strain is capable of producing greater than about 565 mM lactic acid, and most preferably greater than about 665 mM lactic acid when cultured aerobically in a minimal medium. In some embodiments, the AT yeast strain is capable of producing greater than about 50 grams lactic acid per 100 grams glucose when cultured in the minimal medium comprising glucose as a sole carbon source. In certain embodiments, it is preferred that the AT yeast strain is capable of producing between 50 grams and 85 grams lactic acid per 100 grams glucose, and it is more preferred that the AT yeast strain is capable of producing between about 70 and 85 grams lactic acid per 100 grams glucose, when cultured in minimal medium comprising glucose as a sole carbon source. Preferably lactic acid produced by AT yeast is L-(+) lactic acid. Preferably the AT yeast strain belongs to a genus selected from Saccharomyces, Candida, Schizosaccharomyces, Torulaspora, Kluyveromyces, Zygosaccharomyces and Dekkera. More preferably the AT yeast strain belongs to Saccharomyces, Candida, Schizosaccharomyces, or Kluyveromyces. Still more preferably the AT yeast strain belongs to the genus Saccharomyces, such as Saccharomyces cerevisiae. In certain embodiments, the AT yeast strain can be a Saccharomyces cerevisiae that has a genotype pdc1(−6,−2)::loxP pdc5(−6,−2)::loxP pdc6(−6,−2)::loxP ura3-52 YEpLpLDH. In certain embodiments, the yeast strain can be selected from Kluyveromyces thermotolerans, Zygosaccharomyces bailii, Schizosaccharomyces pombe, and Candida glabrata.

In some embodiments, an AT yeast strain depends on having a C₂ carbon source for growth, thus in some cases an AT yeast strain is capable of growing in a second minimal medium comprising a carbon source consisting essentially of glucose and at least one C₂ carbon source. In certain embodiments, an AT yeast strain can be C₂ carbon source-independent (e.g., a CI yeast strain). The CI yeast strain can, in certain embodiments, be capable of growing in a second minimal medium comprising at least one defined carbon source selected from the group consisting of glucose, sucrose, fructose, maltose, lactose, and galactose. In certain embodiments, a CI yeast is capable of growing in a second minimal medium with glucose as the sole carbon source.

An AT yeast strain can be capable of growing in a second minimal medium consisting essentially of at least one defined carbon source, at least one defined nitrogen source, monopotassium phosphate, magnesium sulfate, copper sulfate, ferric chloride, manganese sulfate, sodium molybdate, zinc sulphate, biotin, inositol, thiamine, and water, in certain embodiments. The defined nitrogen source can comprise at least one compound selected from the group consisting of urea, ammonium phosphate, ammonium nitrate, and ammonium sulfate.

In certain embodiments, the exogenous lactate dehydrogenase gene that is part of the genome of an AT yeast strain can be a Lactobacillus plantarum, bovine, Lactobacillus casei, Bacillus megaterium, Rhizopus oryzae, or Bacillus stearothermophylus lactate dehydrogenase gene. Examples of nucleotide sequences of such genes are available on Genbank under accession numbers AJ293008, NP 776524, M76708, M22305, Q9P4B6, and M19396, respectively. In some embodiments, the exogenous lactate dehydrogenase gene is a Lactobacillus plantarum lactate dehydrogenase gene. Preferably the exogenous LDH gene is a L-lactate dehydrogenase gene. Preferably, the exogenous lactate dehydrogenase gene is functionally linked to a promoter. The promoter is preferably a strong, constitutive promoter. In certain embodiments, the preferred promoter is a promoter selected from the group consisting of triose phosphate isomerase promoters, pyruvate decarboxylase promoters, alcohol dehydrogenase promoters, and L-threonine dehydrogenase promoters. It is preferred that the promoter is a triose phosphate isomerase promoter. In certain embodiments, the promoter can be a pyruvate decarboxylase promoter, such as a Kluyveromyces pyruvate decarboxylase promoter.

Certain embodiments of the present invention are directed to an acid-tolerant (AT) S. cerevisiae that produces essentially no ethanol when cultured in a culture medium, whose genome comprises an exogenous lactate dehydrogenase gene that is capable of being expressed in the AT S. cerevisiae. In certain embodiments, the AT S. cerevisiae has no detectable amount of pyruvate decarboxylase activity. Preferably the exogenous LDH gene is a L-lactate dehydrogenase gene. The lactate dehydrogenase protein resulting from the expression has lactate dehydrogenase activity, and the AT S. cerevisiae is capable of producing lactic acid in a minimal medium at a lower pH than its parent S. cerevisiae strain. Preferably the exogenous lactate dehydrogenase gene is a Lactobacillus plantarum lactate dehydrogenase gene. Preferably at least one plasmid in the AT S. cerevisiae comprises the exogenous lactate dehydrogenase gene. In certain embodiments, the AT S. cerevisiae can have a genotype pdc1(−6,−2)::loxP pdc5(−6,−2)::loxP pdc6(−6,−2)::loxP ura3-52 YEpLpLDH. Preferably the AT S. cerevisiae is capable of producing greater than about 500 mM lactic acid in a culture broth, when cultured aerobically in a second minimal medium. Preferably the second culture broth has a pH between about 2.3 and 2.4.

Certain embodiments of the present invention are directed to a recombinant yeast strain having a genome comprising an exogenous lactate dehydrogenase gene that is capable of being expressed in the recombinant yeast strain. Preferably the exogenous LDH gene is a L-lactate dehydrogenase gene. The lactate dehydrogenase protein resulting from the expression has lactate dehydrogenase activity, and the recombinant yeast strain is capable of producing at least about 565 mM lactic acid when cultured in a minimal medium, more preferably at least about 665 mM. The recombinant yeast strain is capable of producing lactic acid at a pH of less than about 3.5, preferably less than about 2.8, more preferably less than about 2.3, and most preferably less than about 2.0. In certain embodiments, the wild type strain of the recombinant yeast strain is Crabtree positive. It is preferred that the recombinant yeast is a S. cerevisiae.

Certain embodiments of the present invention are directed to acid-tolerant C₂ carbon source-independent (CI) yeast strains. CI yeast produce essentially no ethanol when cultured in a culture medium, and their genomes comprise an exogenous lactate dehydrogenase gene that is capable of being expressed. Preferably the exogenous LDH gene is a L-lactate dehydrogenase gene. The lactate dehydrogenase protein produced by expression has lactate dehydrogenase activity. The CI yeast strains are capable of producing lactic acid when cultured under aerobic conditions in a first minimal medium comprising glucose as a sole carbon source, and they are capable of producing lactic acid in the first minimal medium at a lower pH than a parent strain. Preferably the parent strain is C₂ carbon source dependent. In some embodiments the CI yeast has no detectable amount of pyruvate decarboxylase activity. In certain embodiments, a wild type yeast strain of the same strain is Crabtree positive. In some embodiments, a CI yeast strain can comprise a Lactobacillus plantarum, bovine, Lactobacillus casei, Bacillus megaterium, Rhizopus oryzae, or Bacillus stearothermophylus exogenous lactate dehydrogenase gene. Preferably the CI yeast strain comprises a Lactobacillus plantarum lactate dehydrogenase gene. A CI yeast strain chromosome can comprise the exogenous lactate dehydrogenase gene and/or at least one plasmid comprising an exogenous lactate dehydrogenase gene can be present in the CI yeast strain. In certain embodiments, an exogenous lactate dehydrogenase gene can be a part of a 2 micron plasmid.

In certain embodiments, a CI yeast strain is capable of producing lactic acid at a pH of less than about 2.8, more preferably at a pH less than about 2.3. In some embodiments the CI yeast strain is capable of producing greater than about 50 g lactic acid/100 g glucose when cultured in minimal medium; in some embodiments, between about 50 and 85 g lactic acid/100 g glucose; and in some embodiments between about 70 and 85 g lactic acid/100 g glucose. In some embodiments, a CI yeast strain is capable of producing greater than about 565 mM lactic acid in a culture broth, when cultured aerobically in a minimal medium. Preferably the CI yeast strain is cultured at a pH between about 2.3 and 2.4. More preferably, the CI yeast strain is capable of producing greater than about 665 mM lactic acid. It is preferred that a CI yeast strain belongs to a genus selected from Saccharomyces, Candida, Schizosaccharomyces, Torulaspora, Kluyveromyces, Zygosaccharomyces and Dekkera. More preferably, a CI yeast strain belongs to a genus selected from the group consisting of Saccharomyces, Candida, Schizosaccharomyces, and Kluyveromyces. In some embodiments, the CI yeast strain belongs to a species selected from the group consisting of Saccharomyces cerevisiae, Kluyveromyces thermotolerans, Zygosaccharomyces bailii, Schizosaccharomyces pombe, and Candida glabrata. A CI yeast strain can be a Saccharomyces cerevisiae that has a genotype pdc1(−6,−2)::loxP pdc5(−6,−2)::loxP pdc6(−6,−2)::loxP ura3-52 YEpLpLDH, in certain embodiments. The CI yeast strain can be capable of growing in an aerobic batch culture, an aerobic fed-batch culture, or an aerobic chemostat.

The CI yeast strain can be capable of growing in a second minimal medium comprising at least one defined carbon source selected from the group consisting of glucose, sucrose, fructose, maltose, lactose, and galactose. Certain CI yeast strains are capable of growing in a minimal medium consisting essentially of at least one defined carbon source selected from the group consisting of glucose, sucrose, fructose, maltose, lactose, and galactose, at least one of nitrogen source selected from the group consisting of urea, ammonium phosphate, ammonium nitrate, and ammonium sulfate; monopotassium phosphate, magnesium sulfate, copper sulfate, ferric chloride, manganese sulfate, sodium molybdate, zinc sulphate, biotin, inositol, thiamine, and water.

Certain embodiments of the present invention are directed to minimal culture media that comprise a base medium consisting essentially of at least one defined carbon source, at least one nitrogen source, monopotassium phosphate, magnesium sulfate, copper sulfate, ferric chloride, manganese sulfate, sodium molybdate, zinc sulphate, biotin, inositol, thiamine, and water. In certain embodiments, the minimal culture medium consists essentially of the base medium. In certain embodiments, the defined carbon source comprises a C₂ carbon source and optionally at least one compound selected from the group consisting of glucose, sucrose, fructose, lactose, galactose, and maltose. Alternatively, in some embodiments, the minimal culture medium comprises glucose as the sole carbon source. In certain embodiments, the nitrogen source is a compound selected from the group consisting of urea, ammonium sulfate, ammonium nitrate and ammonium phosphate. The minimal culture medium comprises between about 0.5 and 5 g ammonium sulfate/liter in some embodiments; more preferably between about 0.5 and 2 g ammonium sulfate/liter; and most preferably between about 1 and 2 g ammonium sulfate/liter. In certain embodiments, the minimal culture medium comprises between about 0.1 and 2 g urea/liter; more preferably between about 0.1 and 1 g urea/liter; and most preferably between about 0.5 and 2 g urea/liter. The minimal culture medium can, in certain embodiments, comprise calcium carbonate. The minimal medium can comprise about 2.78 g/liter calcium carbonate, in some embodiments. In some embodiments, the minimal culture medium comprises about 1000 ppm Ca⁺². In certain embodiments, the minimal medium comprises between about 5 g and 100 g glucose/liter. In certain embodiments, the minimal culture medium comprises between about 0.2 and 2 g monopotassium phosphate/liter; between about 0.1 and 1 g magnesium sulfate/liter; between about 5 and 50 micrograms copper sulfate/liter; between about 0.05 and 0.25 mg ferric chloride/liter; between about 0.05 and 0.5 mg manganese sulfate/liter; between about 0.05 and 0.25 mg sodium molybdate/liter; between about 0.05 and 0.5 mg zinc sulphate/liter; between about 0.5 and 2.5 micrograms biotin/liter; between about 0.5 and 4 mg inositol/liter; and between about 0.05 and 0.5 mg thiamine/liter.

In some embodiments, a minimal culture medium of the present invention can comprise between about 5 g glucose/liter and 100 g glucose/liter or between about 0.1 and 1 wt % ethanol, about 5 g ammonium sulfate/liter or about 1 g urea/liter, about 1 g monopotassium phosphate/liter, about 0.5 g magnesium sulfate/liter, about 40 micrograms copper sulfate/liter, about 0.2 mg ferric chloride/liter, about 0.4 mg manganese sulfate/liter, about 0.2 mg sodium molybdate/liter, about 0.4 mg zinc sulphate/liter, about 2 micrograms biotin/liter, 2 mg inositol/liter, and about 0.4 mg thiamine/liter. In certain embodiments, the minimal culture medium can further comprise between about 0.1 wt % ethanol and 1 wt % ethanol.

Certain embodiments of the present invention are directed to culture media consisting essentially of water, about 70 g/liter glucose, about 0.5 wt % ethanol, about 1 g/liter urea, about 1 g/liter monopotassium phosphate, about 0.5 g/liter magnesium sulfate heptahydrate, about 2.78 g/liter calcium carbonate, about 62.5 micrograms/liter copper sulfate pentahydrate, about 200 micrograms/liter ferric chloride, about 450 micrograms/liter manganese sulfate monohydrate, about 235 micrograms/liter sodium molybdate dihydrate, about 712 micrograms/liter zinc sulfate heptahydrate, 2 micrograms/liter biotin, 2000 micrograms/liter inositol, and 400 micrograms/liter thiamine hydrochloride.

Some embodiments of the present invention are directed to culture media comprising between about 400 and 1100 ppm N, between about 215 and 287 ppm K⁺, between about 525 and 700 ppm PO₄⁻², about 49 ppm of Mg⁺², about 195 ppm SO₄⁻², about 1100 ppm of Ca⁺², about 0.07 ppm Fe⁺³, about 0.145 ppm Mn⁺², about 0.09 ppm Mo⁻⁴, about 0.16 ppm Zn⁺², about 0.015 ppm Cu⁺², about 0.002 mg/liter biotin, about 2 mg/liter inositol, about 0.4 mg/liter thiamine hydrochloride, and water.

Certain embodiments of the present invention are directed to recombinant yeast strains having a genome comprising an exogenous lactate dehydrogenase gene that is capable of being expressed in the recombinant yeast strain. The lactate dehydrogenase protein resulting from the expression has lactate dehydrogenase activity, and when the recombinant yeast strain is cultured in minimal medium comprising glucose as the sole carbon source it is capable of producing at least about 50 grams lactic acid/100 grams glucose. Preferably the recombinant yeast strain is capable of producing between about 50 and 85 grams lactic acid/100 grams glucose, and most preferably the recombinant yeast strain is capable of producing between about 70 and 85 grams lactic acid/100 grams glucose when grown in minimal medium having glucose as a sole carbon source. The recombinant yeast strain is capable of growing at a pH of less than about 3.5, more preferably at a pH less than about 2.8, still more preferably at a pH less than about 2.3, and most preferably at a pH less than about 2.

Certain embodiments of the present invention are directed to methods of producing lactic acid involving aerobically culturing in a first culture medium a recombinant yeast strain having a genome comprising an exogenous lactate dehydrogenase gene that is capable of being expressed in the recombinant yeast strain, wherein a protein resulting from the expression has lactate dehydrogenase activity, wherein the recombinant yeast strain is capable of producing at least about 50 grams lactic acid/100 grams glucose when grown in a minimal medium comprising glucose as the sole carbon source, and wherein the recombinant yeast strain is capable of growing at a pH of less than about 3.5.

Certain embodiments of the present invention are directed to an acid tolerant (AT) yeast strain that is recovered by a selection process that involves growing a first yeast strain (e.g., parent strain) in a first aerobic culture. The first aerobic culture is started by inoculating a first minimal medium with a first yeast strain that produces essentially no ethanol when cultured in a culture medium, and that comprises a genome having an exogenous lactate dehydrogenase gene that is capable of being expressed. In certain embodiments, the first yeast strain lacks at least one of pyruvate decarboxylase activity or alcohol dehydrogenase activity. The lactate dehydrogenase protein that results from the expression has lactate dehydrogenase activity. In certain embodiments, a wild type yeast strain for the first yeast strain is Crabtree positive. During the growth of the first aerobic culture, the pH of the culture decreases. The selection process further comprises the step of determining about the lowest pH at which the first yeast strain is still capable of growing in the first minimal medium, and the step of recovering at least one second yeast strain from the first aerobic culture, when the first aerobic culture is still growing, and the pH is about at its lowest. The selection process can, in certain embodiments, further comprise the step (1) of growing a second aerobic culture that is started by inoculating fresh minimal medium with the recovered second yeast strain. During the growth of the second aerobic culture the pH of the culture decreases. Next (2) at least one third yeast strain is recovered from the second aerobic culture when the second aerobic culture is still growing, and the pH of the culture is less than about the lowest pH of the first aerobic culture. In some embodiments, steps (1) and (2) are repeated at least one time involving inoculating the fresh minimal medium with a yeast strain recovered from the previous repetition. Preferably, the about lowest pH of the aerobic culture at which the AT yeast strain is growing during the last repetition is less than about the lowest pH of the aerobic culture at which the AT yeast strain was growing in the previous repetition.

Certain embodiments of the present invention are directed to an acid-tolerant C₂ carbon source-independent (CI) yeast strain selected by a process comprising inoculating a minimal medium with an AT yeast strain that requires the minimal medium to comprise a C₂ carbon source for its growth. The yeast strain is cultured in a series of aerobic batch cultures using a second minimal medium. At the start of the series, the second minimal medium comprises glucose and a C₂ carbon source as the sole carbon sources and at concentrations sufficient to permit growth of the yeast culture. Over the series of batch cultures the concentration of the C₂ carbon source is decreased, and each successive batch culture is seeded with yeast grown in a batch culture from earlier in the series. At least one CI yeast strain is recovered from the series of batch cultures that is capable of growing without a C₂ carbon source and with glucose as a sole carbon source. In certain embodiments, the AT strain lacks at least one of pyruvate decarboxylase enzyme activity or alcohol dehydrogenase enzyme activity. In some embodiments the AT strain can be Crabtree positive.

Some embodiments of the present invention are directed to methods of producing lactic acid or salts thereof. The methods involve culturing an AT yeast strain or a CI yeast strain in a minimal medium. The yeast strains and the minimal medium are as described above. In some embodiments, a culture broth resulting from the culturing of the AT or CI yeast strain comprises less ppm of at least one of glycerol, erythritol, malic acid, pyruvic acid, succinic acid, formic acid, and fumaric acid than a culture broth resulting from the culturing of the parent strain in essentially the same minimal medium under essentially the same culture conditions.

The culture medium that the AT or CI yeast strain is cultured in to produce the lactic acid and salts thereof can be a minimal medium comprising at least one defined carbon source selected from the group consisting of glucose, sucrose, fructose, maltose, lactose, and galactose. In certain embodiments, in which the yeast strain is C₂ carbon source independent, the minimal medium comprises glucose as the sole carbon source. In other embodiments an AT yeast strain is C₂ carbon source-dependent and the minimal medium comprises a carbon source consisting essentially of glucose and at least one C₂ carbon source. In certain embodiments the minimal culture medium consists essentially of at least one defined carbon source, at least one nitrogen source, monopotassium phosphate, magnesium sulfate, copper sulfate, ferric chloride, manganese sulfate, sodium molybdate, zinc sulphate, biotin, inositol, thiamine, and water, wherein the nitrogen source is selected from the group consisting of urea, ammonium sulfate, ammonium phosphate, and ammonium nitrate. In some embodiments, lactic acid is recovered and purified from the resulting culture broth. The purification can comprise at least one of distillation, ion exchange, nanofiltration or solvent extraction.

In some embodiments, the culture broth resulting from the culturing comprises greater than about 500 mM lactic acid, more preferably 565 mM lactic acid, and most preferably greater than about 665 mM lactic acid. In certain embodiments, the AT or CI yeast strain can be capable of producing lactic acid at a pH of less than about 3.5, more preferably at a pH of less than about 2.8, and most preferably at a pH of less than about 2.3. Preferably the lactic acid produced consists essentially of L-lactic acid. In certain embodiments, the AT or CI yeast strain can belong to a genus selected from the group consisting of Saccharomyces, Candida, Schizosaccharomyces, and Kluyveromyces, more preferably the AT or CI yeast strain can be a Saccharomyces cerevisiae. In certain embodiments, the AT or CI yeast strain can be a Saccharomyces cerevisiae that has a genotype pdc 1 (−6,−2)::loxP pdc5(−6,−2)::loxP pdc6(−6,−2)::loxP ura3-52 YEpLpLDH. The AT or CI yeast strain can be cultured in an aerobic batch culture, aerobic fed-batch culture, or an aerobic chemostat. The culture broth resulting from the culturing of the AT yeast strain can, in certain embodiments, comprise less ppm of at least one of glycerol, erythritol, malic acid, pyruvic acid, succinic acid, formic acid, and fumaric acid basis than a culture broth resulting from the culturing of its parent strain in essentially the same minimal medium under essentially the same culture conditions. The method of producing lactic acid or salts thereof, can further comprise the step of purifying the culture broth by, for example, using at least one of distillation, ion exchange, nanofiltration or solvent extraction.

An embodiment of the present invention is directed to an acid-tolerant yeast strain having a deposit number NRRL Y-30696. Another embodiment of the present invention is directed to acid-tolerant C₂ carbon source-independent yeast strains having deposit numbers NRRL Y-30697 and Y-30698.

Certain embodiments are directed to a fermentation brothcomprising at least about 500 mM lactic acid and a first group of compounds. More preferably the broth comprises at least about 565 mM lactic acid, and most preferably at least about 665 mM lactic acid. The ratio of the mM lactic acid to mM of the first group of compounds in the fermentation is at least about 54, more preferably at least about 66, and most preferably at least about 184. The first group of compounds consists of glycerol, erythritol, mannitol, malic acid, pyruvic acid, succinic acid, formic acid, and fumaric acid. Preferably the fermentation broth has a pH between about 2.3 and 2.4. Preferably the fermentation broth is a product of the fermentation of a S. cerevisiae, and more preferably a recombinant S. cerevisiae, as described above. The culturing that produces the fermentation can be performed in an aerobic batch culture, aerobic fed-batch culture, or in an aerobic chemostat.

Certain embodiments of the present invention are directed to a plasmid comprising a replication origin and a Lactobacillus lactate dehydrogenase gene functionally linked to a promoter. The replication origin is preferably a yeast replication origin known in the art, such as a 2 micron replication origin. Preferably the lactate dehydrogenase gene is a L-lactate dehydrogenase gene. The Lactobacillus lactate dehydrogenase gene can be any that can be expressed in yeast when functionally linked to a promoter. Preferably the lactate dehydrogenase gene is a Lactobacillus plantarum lactate dehydrogenase gene. The promoter can be any known in the art recognized by a yeast. Preferably the promoter is recognized by S. cerevisiae. In certain embodiments, the promoter can be a triose phosphate isomerase promoter. In certain embodiments, the promoter can be a pyruvate decarboxylase promoter, such as a Kluyveromyces pyruvate decarboxylase promoter. In other embodiments, the promoter is selected from alcohol dehydrogenase promoters, and L-threonine dehydrogenase promoters.

Development of cost effective production processes for lactic acid, are desirable for commodity applications. It is desirable to have strains of Saccharomyces cerevisiae, among other yeasts, which do not produce ethanol but that do produce lactic acid. Such yeasts that are capable of producing lactic acid in chemically defined minimal media, in a low pH environment, and that do not require C₂ compounds for growth are desirable.

### BRIEF DESCRIPTION OF THE DRAWINGS

FIG. 1 is a process flow diagram of an embodiment of the present invention.

FIG. 2 is a plasmid map for YEpLpLDH FIG. 3 is a graph of lactic acid production by a Pdc negative S. cerevisiae strain comprising an exogenous lactate dehydrogenase gene.

FIG. 4 is a graph of lactic acid production by an acid tolerant S. cerevisiae strain of the present invention.

### DESCRIPTION OF ILLUSTRATIVE EMBODIMENTS

“Pyruvate decarboxylase” (Pdcp) refers to any protein (e.g., enzyme), which can catalyze the conversion of pyruvate to acetaldehyde. “PDC” refers to a wild type gene of pyruvate decarboxylase. “pdc” refers to a mutant pyruvate decarboxylase gene. “No detectable amount of pyruvate decarboxylase activity” refers to pyruvate decarboxylase activity in a yeast that is below the detection limit of 0.005 micromole min⁻¹ mg⁻¹ protein when using the methods previously described (van Maris, et al. 2003). Pyruvate decarboxylase activity can be reduced or essentially eliminated from a yeast strain using methods known in the art. For example, a pyruvate decarboxylase structural gene, a pyruvate decarboxylase structural gene's promoter, a gene that regulates the pyruvate decarboxylase structural gene expression, or a promoter of the regulatory gene can be mutated, disrupted, or at least a portion of the gene can be deleted. Still further, the gene expression can be altered using other methods known in the art. For example, an antisense construct can be introduced into a yeast strain that reduces the translation of pyruvate decarboxylase mRNA to pyruvate decarboxylase protein.

“Lactate dehydrogenase” (Ldhp) refers to a protein (e.g., enzyme), which catalyzes the conversion of pyruvate to lactate. “LDH” refers to a wild type gene that when expressed yields a protein that has lactate dehydrogenase activity. “ldh” refers to a mutant lactate dehydrogenase gene. A LDH as used in the present application can include genes that are not named lactate dehydrogenase in the art, when expressed result in protein having lactate dehydrogenase activity. Lactate dehydrogenase genes can be stereospecific. That is, a lactate dehydrogenase gene may catalyze a reaction to produce only L-lactate or only D-lactate. Other lactate dehydrogenases catalyze a reaction to produce both L- and D-lactate. A L-lactate dehydrogenase gene catalyzes the conversion of pyruvate to L-lactate.

“Pdc negative yeast strain” refers to a yeast that has no detectable pyruvate decarboxylase activity, and that does not grow in an aerobic environment on glucose as a sole carbon source in a synthetic culture medium. At least some Pdc negative strains do not produce detectable amounts of ethanol (e.g., less than about 1 ppm) during growth in an aerobic environment in a minimal medium. A Pdc negative Saccharomyces cerevisiae grown in an aerobic glucose-limited chemostat on synthetic medium requires addition of small amounts of a C₂ carbon source (e.g., ethanol, acetaldehyde, and/or acetate). The isogenic wild type strain of the Pdc negative strain is Crabtree positive and has detectable pyruvate decarboxylase activity (see discussion below). A Pdc negative strain that is not capable of growing in culture medium comprising glucose as the sole carbon source can be derived when pyruvate decarboxylase activity is eliminated (e.g., by disruption or mutation of the structural genes, or disruption of the regulation of gene expression, among others) from a Crabtree positive wild type strain.

“Wild type yeast” refers to a yeast, which when it has heritable genetic alterations introduced into its genome, results in the production of a mutant yeast. Restated, the mutant yeast strain has a different genotype than its wild type strain in that certain mutations, deletions or insertions have been introduced into its genome that are not present in the wild type yeast strain's genome. Thus, the wild type yeast strain lacks the changes that are present in the genome of the mutant yeast strain. The mutant yeast strain can, in some cases, have a different phenotype than the wild type strain. The mutant yeast strain can be prepared by methods known in the art, including those involving homologous recombination, directed mutagenesis or random mutagenesis, among others. In certain cases, the mutant yeast strain can be recovered by a process involving natural selection.

“Parent yeast” refers to a yeast from which a new yeast strain is derived directly. For example, a parent strain might comprise a yeast with an exogenous lactate dehydrogenase gene in its genome that requires a C₂ carbon source (see below) for growth. An acid tolerant yeast strain having the lactate dehydrogenase gene may be derived from the parent strain through a selection process for acid tolerance. The acid-tolerant C₂ carbon source dependent yeast strain may in turn become the parent strain of an acid-tolerant C₂ carbon source independent yeast strain having the lactate dehydrogenase gene through a selection process for C₂ carbon source independence of the acid-tolerant yeast strain. A parent strain can, in some instances, also be a wild type strain, though this is not a requirement.

“C₂ carbon source-independent yeast strain” refers to a yeast that produces essentially no ethanol and that, when cultured on minimal medium having glucose as the sole carbon source, does not require a C₂ carbon source. The C₂ carbon source-independent yeast strain can be derived through manipulation (e.g., selection or site directed mutagenesis, among others) of a parent strain (that produces essentially no ethanol) that requires a C₂ carbon source to grow in minimal medium in which glucose is the only other carbon source in an aerobic culture.

An “acid tolerant yeast” refers to a yeast that is capable of producing lactic acid at a pH that is lower than its parent strain can. Acid-tolerant yeasts can produce no detectable amount (e.g., less than 1 ppm) of ethanol during growth in an aerobic environment. For S. cerevisiae in a batch culture the pH at which certain acid-tolerant S. cerevisiae of the present invention can produce lactic acid is less than about 4.

“Crabtree effect” is defined as alcoholic fermentation carried out by a yeast strain in (a) an environment comprising excess oxygen and excess sugar (e.g., carbohydrates) (in certain embodiments “excess sugar” is at a concentration above about 1 mM) or (b) a culture in which the specific growth rate of the yeast strain is higher than the critical specific growth rate on glucose (e.g., about two-thirds of the maximum specific growth rate on glucose). A yeast strain is “Crabtree positive,” if it exhibits the Crabtree effect, and a Crabtree positive yeast strain, employs the pyruvate decarboxylase route as its main pyruvate decarboxylation pathway in the presence of excess sugar. Examples of Crabtree positive yeasts can be found among Saccharomyces cerevisiae, Candida glabrata (also known as Torulopsis glabrata, among others), Zygosaccharomyces bailii, and Schizosaccharomyces pombe, among others. A “Crabtree negative yeast strain” uses the pyruvate dehydrogenase complex reaction as its main mechanism of pyruvate decarboxylation. When growing aerobically with excess sugar alcoholic fermentation hardly occurs in Crabtree negative yeast strains and respiratory pyruvate metabolism predominates. Elimination of pyruvate decarboxylase activity in Crabtree negative yeast strains appears to have no effect on aerobic growth on sugars. Examples of Crabtree negative yeasts can be found among Candida utilis, Kluyveromyces marxianus, and Yarrowia lipolytica.

The term “culture medium” refers to a solid or liquid medium comprising sufficient nutrients, including at least one carbon source, on which a microorganism (e.g., yeast) can grow. In chemostat, fed-batch, or batch cultures the medium is a liquid.

“Carbon source” refers to an organic compound (e.g., defined carbon source, such as glucose, among others) or a mixture of organic compounds (e.g., yeast extract), which can be assimilated by a microorganism (e.g., yeast) and used to make new cell material. A mixture of organic compounds can be either a complex carbon source in which the exact components and/or the quantities of organic components are unknown or a defined carbon source that consists of known organic compounds (e.g., glucose, fructose, maltose, among others) in known quantities. In certain instances, a complex carbon source can also serve as a complex nitrogen source. Examples of complex carbon sources include starch, maltodextrose, cellulose hydrolysates, and starch hydrolysates, among others, which have been combined with enzymes to produce glucose. A defined carbon source will preferably be at least about 90 wt % pure, more preferably about 95 wt % pure, and most preferably about 98 wt % pure. For example, if glucose is the sole carbon source, the carbon source will comprise at least about 90% glucose. If glucose and fructose are the components of a defined carbon source, at least about 90% of the carbon source will be glucose and fructose. The defined carbon source will preferably comprise a minimal amount of higher saccharides.

“C₂ carbon source” refers to a carbon source having two carbons. Examples of C₂ carbon sources are acetate, acetaldehyde, and ethanol.

“Minimal media” or “synthetic media” refers to culture media for culturing a microorganism (e.g., yeast) that comprise a nitrogen source, salts, trace elements, vitamins, and a carbon source, which are all defined. The carbon source can comprise at least one of glucose, sucrose, lactose, maltose, galactose, or fructose, among others. A minimal medium comprises non-protein nitrogen source. Synthetic media do not comprise for example, a nutrient source, whose composition is not defined, such as corn steep liquor, yeast extract or peptone, among others, which can be used in complex culture media.

“Capable of growing in a liquid culture medium” refers to the ability of a microorganism (e.g., yeast) that is introduced into a liquid culture medium under appropriate culture conditions (e.g., pH and temperature, among others) to replicate such that the biomass of the culture increases during the growth phase of the culture.

“Culturing in a liquid medium” refers to growth of a microorganism and/or continued accumulation of lactic acid produced by a microorganism in a liquid culture medium.

“Chemostat” refers to a device that allows for a continuous culture of microorganisms (e.g., yeast) in which both specific growth rate and cell number can be controlled independently. A continuous culture is essentially a flow system of constant volume to which medium is added continuously and from which continuous removal of any overflow can occur. Once such a system is in equilibrium, cell number and nutrient status remain constant, and the system is in a steady state. A chemostat allows control of both the population density and the specific growth rate of a culture through dilution rate and alteration of the concentration of a limiting nutrient, such as a carbon or nitrogen source.

It is known in the art that chemostats can be used in selection of mutants of microorganisms. By altering the conditions as a culture is grown in a chemostat (e.g., decreasing the concentration of a secondary carbon source necessary to the growth of the inoculum strain, among others) those microorganisms in the population that are capable of growing faster at the altered conditions will be selected and outgrow microorganisms that do not function as well under the new conditions. Typically such selection requires the progressive increase or decrease of at least one culture component over the course of growth of the chemostat culture.

“Batch culture” refers to a closed culture of microorganisms with growth occurring in a fixed volume of culture medium that is continually being altered by the actions of the growing organisms until it is no longer suitable for growth. In batch culture, all nutrients required for microbial growth are present in the medium before beginning cultivation, except for molecular oxygen in aerobic cultivation. It is known in the art that extended cultivation of microorganisms in batch cultures (e.g., shake flasks) can be used to select for spontaneous mutants that grow relatively well under conditions in which the inoculum strain would not grow, or grows poorly.

“A series of batch cultures” involves growing a first batch culture of a first yeast strain in a culture medium with at least one defined component (i.e., the concentration of a particular carbon source) that is to be altered over the series. An aliquot of the first batch culture that has been grown is used to inoculate a second batch culture. An aliquot of the second batch culture that is grown is then used to inoculated a third culture, and so forth. The number of steps in a series can vary. Over the course of the series of batch cultures, the concentration of the defined component is increased or decreased. Those microorganisms that can grow best under the conditions at a given step (e.g., batch culture) in the series are selected (e.g., outgrowing other microorganisms that do not grow as well under the particular culture conditions) and used as inoculum for the next batch culture. Thus, over the course of the series, microorganisms that can grow under conditions that the first yeast strain cannot, or that grow better than the first yeast strain under the same growth conditions can be selected. It is known in the art to then isolate individual, pure strains from the culture that has been selected. This can be done by streak plating of a small amount of the cultured organisms, or by other methods known in the art.

A “fed-batch cultivation” refers to a culturing technique in which one or more nutrients are supplied into the culture medium in a cultivation vessel or fermenter over the course of cultivation of a microorganism. In contrast to a chemostat culture, the microorganisms are contained during cultivation. In some cases, all nutrients are gradually fed to the fermenter. The time conditions, temperature conditions, pH conditions, aeration conditions, and the rate at which certain nutrients are fed to a fermenter depend on the particular strain that is being used.

“Selection” refers to placing yeast under conditions that favor the growth of cells having a particular genotype or particular genotypes. The particular genotype (often the result of a genetic mutation) confers upon the selected yeast an advantage under certain environmental conditions so that the progeny of the selected yeast are able to outgrow and/or replace the parent.

The term “gene” refers to chromosomal DNA, plasmid DNA, cDNA, synthetic DNA, or other DNA that encodes a peptide, polypeptide, protein, or RNA molecule, and regions flanking the coding sequence involved in the regulation of expression.

“Mutation” refers to any change or alteration in a nucleic acid sequence. Several types exist, including point, frame shift, and splicing. Mutation may be performed specifically or randomly.

“Open reading frame (ORF)” refers to a region of DNA or RNA encoding a peptide, polypeptide, or protein.

The term “promoter” or “promoter region” refers to a DNA sequence that includes elements controlling the production of messenger RNA (mRNA) by providing the recognition site for RNA polymerase and/or other factors necessary for start of transcription at the correct site.

“Plasmid” refers to a circular, extrachromosomal, self-replicating piece of DNA.

The term “genome” encompasses both the chromosome(s) and plasmids within a host cell.

“2 micron plasmid” refers to a yeast cloning vector that is capable of replicating within certain yeast cells (e.g., S. cerevisiae). Certain genes that can be located on the plasmid can be expressed when operably linked on the plasmid to a promoter recognized and used in the yeast host cell (e.g., yeast transformed with the 2 micron plasmid).

“Transcription” refers to the process of producing a complementary RNA from a DNA template.

“Translation” refers to the production of protein from messenger RNA.

“At least a concentration of” refers to a minimum concentration (e.g., g lactic acid/L or mM) that can be reached in a particular yeast culture.

“Lactic acid” as used in the present invention encompasses both undissociated acid and lactic acid salts. Thus, X g lactic acid/100 g glucose, refers to the total amount of undissociated lactic acid and lactate anion combined relative to each 100 g glucose fed to a fermentation. If a fermentation broth has a pH value between about 3.0 and 4.5, there will be a significant amount of lactic acid in the undissociated form. Indeed at a pH of 3.0 the molar ratio of undissociated lactic acid to lactate ion at 25° C. is about 7.0; and at a pH of about 4.5 the ratio at 25° C., is about 0.23. The total amount of undissociated lactic acid present in a solution is a function of both the pH of the solution and the overall concentration of lactic acid in the mixture. The lower the solution pH, the higher the percentage of the lactic acid that is present in its undissociated form. For example, where the medium is equal to the pKₐ of lactic acid (about 3.8), 50% of the lactic acid is present in the undissociated form. At pH 4.2, about 31% of the lactic acid is undissociated and at pH 4.0 and 3.9, about 41% and 47% respectively of the lactic acid is undissociated. The fraction of undissociated lactic acid is even lower at higher pH, 18% at pH 4.5 and 6.6% at pH 5.0.

“Fermentation broth” refers to a broth that is produced when a microorganism (e.g., yeast) is cultured in a liquid fermentation medium. The fermentation broth comprises any unused components of the liquid fermentation medium and any metabolites or products that result from fermentation by the organism.

It should be noted, that certain species names mentioned herein, such as Torulopsis glabrata, can refer to the name given in the species description by Barnet, Payne and Yarrow (1).

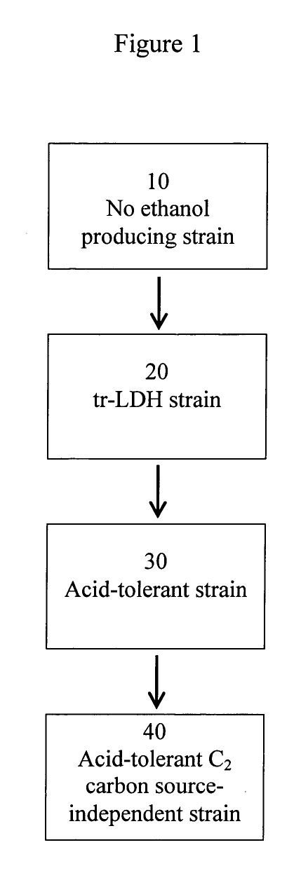

Certain embodiments of the present invention can be better understood with reference to FIG. 1. A yeast strain that produces essentially no ethanol when cultured in a culture medium (e.g., Pdc⁻ yeast strain) 10 can have an exogenous lactate dehydrogenase gene introduced into its genome to yield yeast strain 20. Preferably the exogenous LDH gene is a L-lactate dehydrogenase gene. In certain embodiments the yeast 10 is a Crabtree positive yeast lacking pyruvate decarboxylase activity. In certain embodiments, the yeast 10 belongs to a genus selected from the group consisting of Saccharomyces, Candida, Schizosaccharomyces, Torulaspora, Kluyveromyces, Zygosaccharomyces and Dekkera. Preferably the yeast 10 belongs to a genus selected from the group consisting of Saccharomyces, Candida, Schizosaccharomyces, and Kluyveromyces. Still more preferably the yeast strain belongs to the genus Saccharomyces. The yeast strain can be a strain that belongs to Kluyveromyces thermotolerans, Zygosaccharomyces bailii, Saccharomyces cerevisiae, Schizosaccharomyces pombe, Torulaspora globosa, Torulaspora delbruckii, Dekkera bruxellensis, or Candida glabrata (also known as Torulopsis glabrata). Preferably the yeast strain belongs to Saccharomyces cerevisiae or Candida glabrata, more preferably the yeast strain belongs to Saccharomyces cerevisiae. Preferably the yeast strain belongs to S. cerevisiae, and the yeast strain has a genotype pdc1(−6,−2)::loxP pdc5(−6,−2)::loxP pdc6(−6,−2)::loxP. In some embodiments, the yeast is a Saccharomyces cerevisiae that has a genotype pdc1,5,6 Δ (e.g., partial or complete disruption of the PDC 1, 5, 6 structural genes). Preferably the yeast is non-pathogenic. The yeast is capable of growing in an aerobic batch culture or an aerobic chemostat in an appropriate growth medium.

In some embodiments, the yeast strain 10 can be auxotrophic for ura, leu, or his, among others. In certain embodiments, the yeast strain is ura⁻.

The yeast strain 10 has at least one exogenous lactate dehydrogenase gene (e.g., encoding a protein having lactate dehydrogenase activity) introduced into its genome to produce a tr-LDH (transformed with LDH) yeast strain 20. The introduction of the exogenous lactate dehydrogenase gene can be performed using methods known in the art (e.g., transformation, and electroporation, among others). The genome of the tr-LDH yeast strain 20 comprises the exogenous lactate dehydrogenase gene. That is, at least one chromosome of the yeast strain 20 can comprise at least one exogenous lactate dehydrogenase gene and/or at least one plasmid in the yeast strain can comprise an exogenous lactate dehydrogenase gene. If the yeast strain 10 is auxotrophic, the introduction of the exogenous lactate dehydrogenase gene can be performed such that another gene is introduced at the same time that causes the tr-LDH yeast strain 20 not to be auxotrophic. This can be done using methods known in the art.

As used herein the exogenous lactate dehydrogenase gene can be (1) a gene derived from another organism, (2) a gene derived from the same species or strain (e.g., the parent strain of yeast 10), or (3) a gene of (1) or (2) that has been genetically modified (e.g., codons altered for improved expression in the yeast strain 10, site directed or random mutagenesis, among others). Preferably the exogenous lactate dehydrogenase gene is a Lactobacillus plantarum, bovine, Lactobacillus casei, Bacillus megaterium, Rhizopus oryzae, or Bacillus stearothermophylus modified or unmodified lactate dehydrogenase gene, more preferably it is an unmodified lactate dehydrogenase gene. More preferably the exogenous lactate dehydrogenase gene is a Lactobacillus plantarum lactate dehydrogenase gene. It is preferred that the lactate dehydrogenase gene is a L-lactate dehydrogenase gene.

Preferably the exogenous lactate dehydrogenase gene is functionally linked to a promoter. The promoter can be recognized as such by the yeast strain 10. That is, the promoter can promote transcription of the exogenous lactate dehydrogenase gene in the transformed yeast 20. In certain embodiments, the promoter can be a triose phosphate isomerase promoter (tpi). Other promoters that can be used in certain embodiments include pyruvate decarboxylase promoters, alcohol dehydrogenase promoters, and L-threonine dehydrogenase promoters. Preferably the promoter used is a strong, constitutive promoter in the host organism. In certain embodiments, the promoter linked to the exogenous lactate dehydrogenase gene is a Kluyveromyces pyruvate decarboxylase promoter.

When at least one plasmid in a tr-LDH yeast 20 comprises an exogenous lactate dehydrogenase gene, the plasmid is preferably a high copy number plasmid. In certain embodiments, the plasmid can be a 2 micron plasmid or a low copy number centromeric plasmid. Preferably the plasmid with the exogenous lactate dehydrogenase gene is a 2 micron plasmid having a triose phosphate isomerase promoter (tpi) functionally linked to the exogenous lactate dehydrogenase (LDH) gene. In certain embodiments, a yeast strain 10 (e.g., a Pdc negative S. cerevisiae strain), is transformed (e.g., using methods known in the art) with a 2 micron plasmid comprising a Lactobacillus plantarum L-lactate dehydrogenase gene functionally linked to a tpi promoter. In some embodiments, the lactate dehydrogenase gene is a L-lactate dehydrogenase gene.

Transformation with multi-copy number plasmids can result in there being more than one copy of an exogenous lactate dehydrogenase gene in the genome of a transformed yeast strain 20. In certain embodiments, there can be multiple copies of exogenous lactate dehydrogenase genes introduced (e.g. by homologous recombination or insertion, among others) into the chromosomes of a yeast strain. Multiple copies can be introduced into a single chromosome or copies can be introduced into sites on different chromosomes. In certain embodiments, exogenous lactate dehydrogenase genes can be present in both chromosomes and plasmids within the tr-LDH strain 20.

A tr-LDH yeast strain 20 can undergo selection to produce an acid-tolerant (AT) yeast strain 30. For the selection process a tr-LDH yeast strain 20 is grown aerobically in a minimal medium can comprise at least one of glucose, sucrose, fructose, lactose, galactose, and maltose. In certain embodiments, wherein the tr-LDH yeast strain 20 is C₂ carbon source dependent, the minimal medium can comprise a C₂ carbon source. Preferably the minimal medium comprises glucose and a C₂ carbon source.

In certain embodiments, the culture medium of a batch culture is inoculated with a tr-LDH yeast strain 20. The course of growth of the culture can be monitored along with the pH changes and the amount of lactic acid (and the salts thereof) produced. The lowest pH at which the tr-LDH strain 20 will still grow and produce lactic acid is approximated. A culture of the tr-LDH strain 20 is grown and an aliquot of the culture is removed, when the culture approaches the lowest pH at which the culture is still growing. The aliquot is then used to seed the next batch culture, and an aliquot is removed either at (a) the same low pH as the previous batch culture or (b) at a pH that is lower, and at which the yeast cells are still growing and producing lactic acid. This aliquot can be used to seed a next batch culture. This procedure is repeated over a series, until the pH at which a recovered yeast strain (e.g., acid-tolerant yeast) 30 can grow is lower than the pH at which the tr-LDH parent strain 20 can grow. Like the parent strain (e.g., the Pdc negative tr-LDH yeast strain 20) the acid-tolerant (AT) yeast strain 30 produces essentially no ethanol, and its genome comprises an exogenous lactate dehydrogenase gene that is capable of being functionally expressed in the AT yeast strain 30.

In certain embodiments, an AT yeast strain 30 can be recovered from a chemostat. A chemostat culture that is grown aerobically is started from a tr-LDH strain 20 using a minimal medium. In some embodiments, the minimal medium comprises a C₂ carbon source. The pH of the minimal medium is gradually decreased during culturing of the yeast culture, and an AT yeast strain 30 can be recovered from the chemostat that can grow at a lower pH than the tr-LDH strain 20. In certain embodiments, the AT yeast strain 30 is recovered when the culture reaches about its lowest pH when the yeast in the culture is still growing. The AT yeast strain 30 is as described above.

An AT yeast strain 30 that is C₂ carbon source dependent, selected as described above, can, in certain embodiments, at a pH between 2.3 and 2.4 produce greater than about 500 mM lactic acid, when cultured in a minimal medium comprising a C₂ carbon source and at least one carbon source (e.g., at least one of glucose, sucrose, fructose, maltose, lactose, and galactose). The minimal medium can comprise glucose and a C₂ carbon source as the sole carbon sources, in certain embodiments. In certain embodiments, a C₂ carbon source dependent AT yeast strain 30 can produce greater than 565 mM lactic acid, when cultured in a minimal medium comprising a C₂ carbon source and at least one other carbon source. A C₂ carbon source dependent AT yeast strain can be capable of producing lactic acid at a pH of less than about 3.5, more preferably at a pH less than about 2.8, and most preferably at a pH less than about 2.3.

A series of aerobic batch cultures using a minimal medium can be inoculated with an AT yeast strain that is C₂ carbon source dependent 30. At the start of the series the minimal medium can comprise glucose and a C₂ carbon source as the sole carbon sources and at concentrations sufficient to permit growth of the yeast culture. The concentration of the C₂ carbon source can be decreased over the series of batch cultures, and each successive batch culture can be seeded with yeast grown in a batch culture from earlier in the series. An acid-tolerant C₂ carbon source-independent (CI) yeast strain 40 that is capable of growing without a C₂ carbon source and with glucose as a sole carbon source can be recovered from the series of batch cultures.

Alternatively, the AT yeast strain that is C₂ carbon source dependent 30 can be used to inoculate an aerobic chemostat containing a minimal medium as described above (e.g., comprising a C₂ carbon source), and the concentration of the C₂ carbon source can be decreased over the course of culturing the AT yeast strain in the chemostat. A CI yeast strain 40 can be recovered from the chemostat, once the C₂ carbon source is used up.

Since the CI yeast strain 40 is derived from the AT yeast strain 30 it can comprise an exogenous lactate dehydrogenase gene that is a Lactobacillus plantarum, bovine, Lactobacillus casei, Bacillus megaterium, Rhizopus oryzae, or Bacillus stearothermophylus lactate dehydrogenase gene. The exogenous lactate dehydrogenase gene can reside on a chromosome and/or a plasmid (e.g., a 2 micron plasmid) of the CI yeast strain 40. Preferably the CI yeast strain 40 is capable of producing lactic acid at a pH of less than about 3.5, more preferably at a pH of less than about 2.8, and most preferably at a pH of less than about 2.3. Preferably the CI yeast strain 40 is capable of producing greater than about 565 mM lactic acid in a culture broth, when cultured aerobically in a minimal medium, and more preferably greater than about 665 mM lactic acid. The CI yeast strain 40 can be selected from the group consisting of Saccharomyces cerevisiae, Kluyveromyces thermotolerans, Zygosaccharomyces bailii, Schizosaccharomyces pombe, and Candida glabrata. In certain embodiments the CI yeast strain can be a Saccharomyces cerevisiae that has a genotype pdc1(−6,−2)::loxP pdc5(−6,−2)::loxP pdc6(−6, '2)::loxP ura3-52 YEpLpLDH. The CI yeast strain can be capable of growing in an aerobic batch culture, an aerobic fed-batch culture, or an aerobic chemostat.

One aspect of the present invention relates to the characteristic of Saccharomyces cerevisiae, among other yeasts, being able to grow in chemically defined minimal media. With minimal nutrient input the resulting fermentation broth can contain fewer undesirable, residual organic and inorganic impurities and metabolic intermediates secreted by the organism. Recovering and purifying lactic acid from a fermentation broth that is produced with minimal nutrient input can, in certain embodiments, reduce the production cost dramatically. Thus, it can be preferable to use minimal medium for lactic acid fermentation processes. Nutrients are provided in amounts sufficient to permit growth and metabolic maintenance without excess of the nutrients. The AT and CI yeast can be grown in minimal medium.

A minimal culture medium of the present invention can comprise a base medium consisting essentially of at least one defined carbon source, at least one nitrogen source, monopotassium phosphate, magnesium sulfate, copper sulfate, ferric chloride, manganese sulfate, sodium molybdate, zinc sulphate, biotin, inositol, thiamine, and water. In certain embodiments the minimal culture medium consists essentially of the base medium. When certain C₂ carbon source dependent AT yeast strains are cultured in such a minimal medium, the medium can comprise at least one C₂ carbon source. Other carbon sources that can be part of the medium when culturing either AT or CI strains include glucose, sucrose, fructose, lactose, galactose, and maltose. In certain embodiments, when a CI strain or a C₂ carbon source independent AT strain is being cultured in the minimal medium, glucose may be the sole carbon source.

In certain embodiments, the minimal culture medium comprises water, between about 5 g glucose/liter and 100 g glucose/liter, and in some embodiments the medium further comprises between 0.1 wt % ethanol and 1 wt % ethanol. In certain embodiments, the minimal culture medium further comprises calcium carbonate, preferably about 2.78 g/liter calcium carbonate. In some embodiments the minimal culture medium comprises about 1000 ppm Ca⁺². The nitrogen source of the minimal culture medium can, in some embodiments, be a compound selected from the group consisting of urea, ammonium sulfate, ammonium nitrate, and ammonium phosphate. In some embodiments, the minimal culture medium comprises between about 0.5 and 5 g ammonium sulfate/liter, more preferably between about 0.5 and 2 g ammonium sulfate/liter, and most preferably between about 1 and 2 g ammonium sulfate/liter. In certain embodiments, the minimal culture medium comprises between about 0.1 and 2 g urea/liter, preferably between about 0.1 and 1 g urea/liter, and more preferably between about 0.5 and 2 g urea/liter.

In certain embodiments, the minimal culture medium comprises water, between about 0.2 and 2 g monopotassium phosphate/liter; between about 0.1 and 1 g magnesium sulfate/liter; between about 5 and 50 micrograms copper sulfate/liter; between about 0.05 and 0.25 mg ferric chloride/liter; between about 0.05 and 0.5 mg manganese sulfate/liter; between about 0.05 and 0.25 mg sodium molybdate/liter; between about 0.05 and 0.5 mg zinc sulphate/liter; between about 0.5 and 2.5 micrograms biotin/liter; between about 0.5 and 4 mg inositol/liter; and between about 0.05 and 0.5 mg thiamine/liter.

In some embodiments, the minimal culture medium comprises water, between about 5 g glucose/liter and 100 g glucose/liter or between about 0.1 wt % and 1 wt % ethanol, about 5 g ammonium sulfate/liter or about 1 g urea/liter, about 1 g monopotassium phosphate/liter, about 0.5 g magnesium sulfate/liter, about 40 micrograms copper sulfate/liter, about 0.2 mg ferric chloride/liter, about 0.4 mg manganese sulfate/liter, about 0.2 mg sodium molybdate/liter, about 0.4 mg zinc sulphate/liter, about 2 micrograms biotin/liter, about 2 mg inositol/liter, and about 0.4 mg thiamine/liter.

As explained above, AT or CI yeast can be cultured in a minimal medium to produce lactic acid. The resulting fermentation broth can have a pH between 2.3 and 2.4. In certain embodiments the fermentation broth comprises at least about 500 mM lactic acid and a first group of compounds that consists of glycerol, erythritol, mannitol, malic acid, pyruvic acid, succinic acid, formic acid, and fumaric acid. The ratio of the mM lactic acid to mM of the first group of compounds in the fermentation broth can be at least about 54. Preferably the fermentation comprises at least about 565 mM lactic acid, and more preferably at least about 665 mM lactic acid. In certain embodiments the lactic acid is produced at a pH between 2.3 and 2.4. Preferably the ratio of the mM lactic acid to mM of the first group of compounds is greater than about 66, and more preferably greater than about 184. Preferably a culture broth resulting from the culturing of an AT yeast strain comprises less ppm of at least one of glycerol, erythritol, malic acid, pyruvic acid, succinic acid, formic acid, and fumaric acid than a culture broth resulting from the culturing of its parent strain in essentially the same minimal medium under essentially the same culture conditions. The fermentation broth resulting from the fermentation of an AT or CI yeast can be purified to recover lactic acid using methods known in the art. The purification can involve at least one of distillation, ion exchange, nanofiltration or solvent extraction.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The strains below were used in the examples that follow.

TABLE 1
Saccharomyces cerevisiae strains used

| Strain | Description |
|---|---|
| CEN.PK 113.7D | MATa URA3 PDC1 PDC5 PDC6 |
| | Wild type yeast having pyruvate decarboxylase activity |
| CEN.PK182 | MATa pdc1(−6, −2)::loxP pdc5(−6, −2)::loxP |
| | pdc6(−6, −2)::loxP Pdc negative yeast |
| CEN.PK111-61A | MATa ura3-52 leu2-112 his3-Δ1 |
| | ura⁻yeast |
| RWB837 | MATa pdc1(−6, −2)::loxP pdc5(−6, −2)::loxP |
| | pdc6(−6.−2)::loxP ura3-52 Pdc negative ura⁻ yeast |
| RWB876 | MATa pdc1(−6, −2)::loxP pdc5(−6, −2)::loxP |
| | pdc6(−6.−2)::loxP ura3-52 YEpLpLDH |
| | Pdc negative yeast with exogenous lactate |
| | dehydrogenase activity |
| m850-a | MATa pdc1(−6, −2)::loxP pdc5(−6, −2)::loxP |
| | pdc6(−6.−2)::loxP ura3-52 YEpLpLDH |
| | Pdc negative yeast with exogenous lactate |
| | dehydrogenase activity, acid tolerant |
| Lp4 and Lp4f | MATa pdc1(−6, −2)::loxP pdc5(−6, −2)::loxP |
| | pdc6(−6.−2)::loxP ura3-52 YEpLpLDH |
| | Pdc negative yeast with exogenous lactate |
| | dehydrogenase activity, acid tolerant, |
| | C₂ carbon source independent |

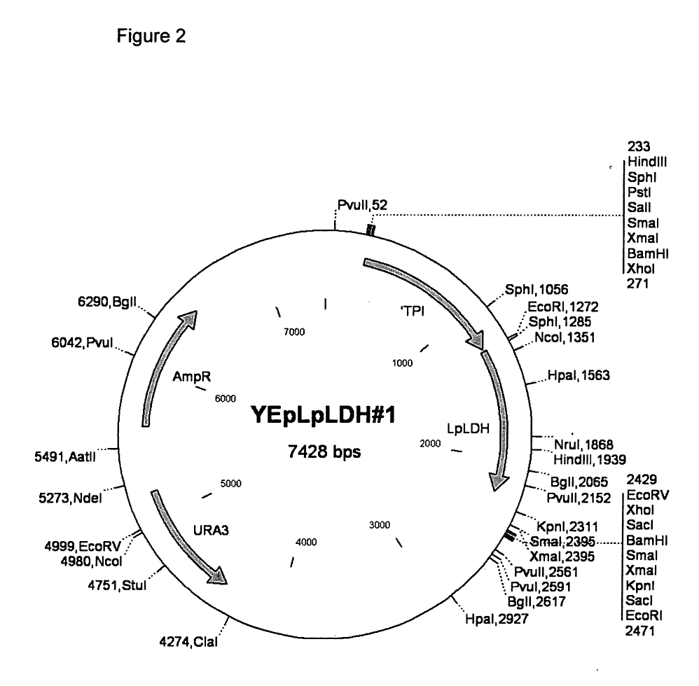

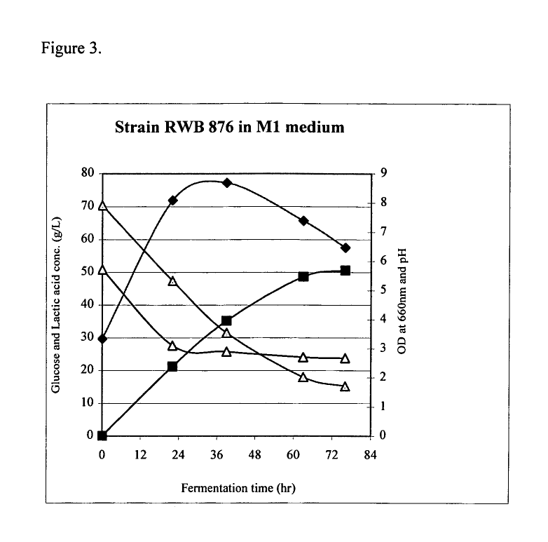

LDH derived from Lactobacillus. plantarum See FIG. 2 for the map of YELpLDH.

In one aspect, the invention discloses and claims fungal cells and cell cultures comprising lactic acid production, acid tolerance, and C₂ carbon source independence as disclosed herein, and in particular, cells of Saccharomyces cerevisiae strains, which comprise RWB876 and its derivatives including m850-a, Lp4, and Lpf4 particularly those that produce lactic acid; those that produce lactic acid and that are also acid tolerant; or those that produce lactic acid, are acid tolerant, and are C₂ carbon source independent.

Such cells and cell cultures may be substantially biologically-pure cultures that comprise, consist essentially of, or consist of a single strain. Illustrative embodiments of the present invention, in the form of biologically-pure cultures of strains
RWB876 (MATa pdc1(−6,−2)::loxP pdc5(−6,−2)::loxP pdc6(−6.−2)::loxP ura3-52 YEpLpLDH) Pdc negative yeast with exogenous lactate dehydrogenase activity;
m850-a (MATa pdc1(−6,−2): :loxP pdc5(−6,−2)::loxP pdc6(−6.−2): :loxP ura3-52 YEpLpLDH) Pdc negative yeast with exogenous lactate dehydrogenase activity, acid tolerant; and

Lp4 and Lp4f (MATa pdc1(−6,−2)::loxP pdc5(−6,−2)::loxP pdc6(−6.−2)::loxP ura3-52 YEpLpLDH) Pdc negative yeast with exogenous lactate dehydrogenase activity, acid tolerant, C₂ carbon source independent have been deposited under conditions that assure that access to the cultures will be available during the pendancy of this patent application to one determined by the Commissioner of Patents and Trademarks to be entitled thereto under 37 C. F. R. § 1.14 and 35 U. S. C. §122. The deposits are available as required by foreign patent laws in countries wherein counterparts of the subject application, or its progeny, are filed. However, it should be understood that the availability of a deposit does not constitute a license to practice the subject invention in derogation of patent rights granted by governmental action.

Further, the subject culture deposits will be stored and made available to the public in accord with the provisions of the Budapest Treaty for the Deposit of Microorganisms, i.e., they will be stored with all the care necessary to keep them viable and uncontaminated for a period of at least five years after the most recent request for the finishing of a sample of the deposit, and in any case, for a period of at least 30 (thirty) years after the date of deposit or for the enforceable life of any patent which may issue disclosing the cultures. The depositor acknowledges the duty to replace the deposits should the depository be unable to furnish a sample when requested, due to the condition of the deposits. All restrictions on the availability to the public of the subject culture deposits will be irrevocably removed upon the granting of a patent disclosing them.

Cultures RW876, m850-a, Lp4 and Lp4f were deposited in the permanent collection of the Northern Regional Research Center (NRRL), Agricultural Research Service Culture Collection, National Center for Agricultural Utilization Research, US Department of Agriculture, 1815 North University Street, Peoria, Ill. 61604, U.S.A. on Oct. 21, 2003 under the terms of the Budapest Treaty, and accorded the accession numbers Y-30696, Y-30697, and Y-30698, respectively. CEN.PK1 13.7D, CEN.PK 182, CEN.PK 11 1-61A, RWB837, were deposited previously and have the accession numbers NRRL Y-30646, NRRL Y-30647, NRRL Y-30648, and NRRL Y-30649, respectively.

### EXAMPLE 1

### Shake Flask Production of Lactic Acid in Chemically Defined Minimal Medium

Fermentations were carried out using strain RWB876, and M1 medium whose composition is listed in Table 2 below.

TABLE 2
M1 Culture Medium
Carbon Sources

| | | |
|---|---|---|
| Glucose | 70 | g/L |

| | |
|---|---|
| Ethanol | 0.5% |

Nitrogen Source and Salts

| | | |
|---|---|---|
| Urea | 1.0 | g/L |
| KH₂PO₄ | 1.0 | g/L |
| MgSO₄.7H₂O | 0.5 | g/L |
| CaCO₃ | 2.78 | g/L |

Trace element solution

| | | |
|---|---|---|
| CuSO₄.5H₂O | 62.5 | μg/L |
| FeCl₃ | 200 | μg/L |
| MnSO₄.H₂O | 450 | μg/L |
| Na₂Mo₄.2H₂O | 235 | μg/L |
| ZnSO₄.7H₂O | 712 | μg/L |

Vitamins

| | | |
|---|---|---|
| Biotin | 2 | μg/L |
| Inositol | 2000 | μg/L |
| Thiamine HCl | 400 | μg/L |

A 50% glucose stock solution was prepared and autoclaved separately from other components of the medium, and the glucose solution was ultimately added to the medium to obtain the final 70 g/liter concentration. Ethanol was added to the cooled autoclaved medium. A source of Ca⁺² was used in the medium to better maintain the cells in an active, physiological stage. In this example, a total of 1112 ppm Ca⁺² were added. The pH of the M1 medium was not adjusted.

The fermentations were carried out in 250-ml triple baffled shake flasks containing 100-ml (final volume) M1 medium. Fermentation was carried out at 32° C. with shaking at 180-rpm in a New Brunswick G-25 shaker. The results of the fermentation of strain RW876 in M1 medium are depicted in FIG. 3. The pH of the RW876 culture continued to decrease while lactic acid concentration increased. The organism was capable of growth at a pH of about 3.0, however as the pH decreased further, the growth of the culture ceased, and the cells began to die off. This cell death was indicated by the decreasing cell density measured at OD₆₆₀ₙₘ.

S. cerevisiae cells do not typically grow in culture medium at a pH that is lower than about 3.0. Cells that can continue to produce lactic acid at low pH (e.g., as lactic acid accumulates) are desirable. Acid-tolerant cells that continue to grow at a pH lower than 3.0 were selected by transferring surviving cells from the end of a fermentation in which the pH of the medium had decreased to 2.80 into fresh medium. This transferring process was repeatedly carried out and the pH at transfer decreased progressively from 2.80 to 2.70 and finally to 2.60. After each transfer the surviving cells were allowed to grow and produce lactic acid for up to 48-hrs. A total of twenty one consecutive transfers were carried out to obtain the acid-tolerant mutant called m850-a. Another five consecutive transfers were performed to stabilize the mutant.

### EXAMPLE 2

### Comparison of Fermentation by Acid-Tolerant Mutant, m850-a, and its Parent Strain RWB876

In this example the lactic acid production ability of the acid-tolerant mutant m850-a grown in M1 medium was compared to that of its parent strain RWB876. Fermentations were carried out in shake flasks in M1 medium using the conditions described in Example 1. Results are summarized in Table 3 below. The ability of the acid-tolerant mutant m850-a to continue growing at a pH less than about 3.0, allowed it to accumulate a higher concentration of lactic acid in its fermentation broth than its parent strain, RW876.

TABLE 3

| | Fermentation of RWB876 | | | | Fermentation of m850-a | | | |
|---|---|---|---|---|---|---|---|---|
| | | | glu. | lact. | | | glu. | lact. |
| time (hr) | OD | pH | (g/L) | (g/L) | OD | pH | (g/L) | (g/L) |
| 0 | 3.34 | 5.72 | 70.40 | 0 | 3.36 | 5.70 | 71.70 | 0 |
| 22 | 8.09 | 3.11 | 47.36 | 21.24 | 7.18 | 3.07 | 44.45 | 23.78 |
| 39 | 8.69 | 2.90 | 31.58 | 35.24 | 8.57 | 2.78 | 25.87 | 42.13 |
| 63 | 7.39 | 2.71 | 18.02 | 48.65 | 9.31 | 2.65 | 7.52 | 58.54 |
| 76 | 6.47 | 2.68 | 15.23 | 50.56 | 9.00 | 2.60 | 3.34 | 62.35 |

*glu. = glucose; lact. = lactic acid

### EXAMPLE 3

### Comparison of Acid-Tolerant Mutant, m850-a, with its Parent Strain RWB876 when Both are Grown in M1 Medium with an Initial Low pH

The experiment was carried out in shake flasks using M1 medium under the conditions described in Example 1, except that the initial pH of the medium was adjusted to 3.50 and no CaCO₃ was added to the medium. The results are summarized in Table 4. Both strains were capable of initiating growth at pH 3.50 in the absence of CaCO₃. At low pH, the acid-tolerant mutant, m850-a, demonstrated the ability to grow and to produce a higher concentration of lactic acid in fermentation broth than its parent strain.

TABLE 4

| | Fermentation of RWB876 | | | | Fermentation of m850-a | | | |
|---|---|---|---|---|---|---|---|---|
| | | | glu. | lact. | | | glu. | lact. |
| time (hr) | OD | pH | (g/L) | (g/L) | OD | pH | (g/L) | (g/L) |
| 0 | 3.04 | 3.50 | 73.80 | 0 | 3.00 | 3.50 | 73.50 | 0 |
| 21 | 5.85 | 2.43 | 57.00 | 11.45 | 6.04 | 2.41 | 56.40 | 12.60 |
| 31 | 6.54 | 2.36 | 54.61 | 16.35 | 6.76 | 2.34 | 50.61 | 18.24 |
| 44 | 6.68 | 2.34 | 50.45 | 20.76 | 7.20 | 2.31 | 45.40 | 24.52 |
| 68.5 | 6.67 | 2.33 | 45.12 | 24.12 | 8.16 | 2.27 | 38.16 | 30.36 |
| 79.5 | 6.69 | 2.33 | 43.67 | 24.99 | 6.72 | 2.27 | 36.15 | 32.62 |

*glu. = glucose; lact. = lactic acid

### EXAMPLE 4

### Analysis of the Fermentation Broths of Example 2

The ultimate cost of producing polymer grade lactic acid is associated with the costs associated with removing impurities produced in the fermentation broth. The fermentation broths of Example 2 were analyzed for concentration of lactic acid and concentration of certain impurities.

The nutrient input (excluding ethanol and glucose) for the fermentations carried out in Example 2 was 2.504 g/liter with the return of 557.9 mM and 686.4 mM lactic acid for fermentations of strain RWB876 and strain m850-a, respectively. The HPLC analysis of the final fermentation broths (e.g., that of RWB876 and m850-a) for polyols and organic acids are summarized in Table 5. Fermentation by RWB876 resulted in the production of 557.9 mM lactic acid, and a total of 15.634 mM of polyols and other organic acids. The fermentation broth for strain m850-a yielded a total of 12.740 mM of polyols and other organic acids, and 686.4 mM lactic acid. At least partial removal of these fermentation by-products (e.g., polyols and organic acids) and certain unused components of medium are necessary to obtain a higher purity (e.g., polymer-grade) lactic acid. The impurities produced by strains of the present invention during lactic acid fermentation are lower than those produced by certain lactic acid-producing, recombinant E. coli strains known in the art (Chang, et al. 1999).

TABLE 5

| | Glucose | Lactic | Glycerol | Erythritol | Mannitol | Malic acid | Pyruvic acid | Succinic acid | Formic acid | Fumaric |
|---|---|---|---|---|---|---|---|---|---|---|
| Strain | (g/L) | acid (g/L) | (PPM) | (PPM) | (PPM) | (PPM) | (PPM) | (PPM) | (PPM) | acid (PPM) |
| RWB 876 | 15.10 | 50.21 | 895 | 114 | 0 | 20 | 214 | 198 | 32 | 2 |
| m850-a | 3.38 | 61.78 | 798 | 123 | 0 | 21 | 121 | 78 | 39 | 2 |

### EXAMPLE 5

### PDC Negative S. cerevisiae Mutants Capable of Utilizing Glucose as the Sole Carbon Source (C₂-Independent) for Cell Growth and for Lactic Acid Production

This experiment was carried out in shake flasks using M1 medium under the conditions described in Example 1 except that the 0.5% ethanol was omitted. Two acid tolerant C₂ carbon source independent S. cerevisiae strains were isolated from m850-a, which is C₂ carbon source dependent. The two strains were isolated using a series of batch cultures in which the concentration of the C₂ carbon source was reduced over the series. As illustrated in the Table 6, two mutant strains were isolated (e.g., Lp4 (NRRL Y-30697) and Lp4f (NRRL Y-30698)), that were capable of utilizing glucose as the sole carbon source for both growth and lactic acid production. Lp4 was isolated after 30 transfers and Lp4f was isolated after about 45 transfers. Lp4 is less acid tolerant than Lp4f. These mutants retained both their ability to grow and their ability to produce lactic acid at a low pH in defined medium, even though the medium did not comprise ethanol.

TABLE 6

| | Fermentation of Lp4 | | | | Fermentation of Lp4f | | | |
|---|---|---|---|---|---|---|---|---|
| | | | glu. | lact. | | | glu. | lact. |
| time (hr) | OD | pH | (g/L) | (g/L) | OD | pH | (g/L) | (g/L) |
| 0 | 3.56 | 5.65 | 67.60 | 0 | 3.48 | 5.57 | 67.60 | 0 |
| 24 | 8.16 | 3.44 | 51.80 | 13.01 | 7.68 | 3.28 | 47.20 | 17.34 |
| 44 | 10.48 | 3.08 | 34.64 | 25.72 | 8.96 | 2.90 | 23.70 | 37.32 |
| 62.5 | 11.20 | 2.96 | 23.70 | 35.24 | 9.04 | 2.78 | 8.41 | 50.85 |
| 71.5 | 12.80 | 2.90 | 18.60 | 38.22 | 10.88 | 2.75 | 5.35 | 53.85 |

*glu. = glucose; lact. = lactic acid

### EXAMPLE 6

### Analysis of the Fermentation Broths of Example 5

The nutrient input (excluding glucose) for the fermentations carried out in Example 5 was 2.504 g/liter with the return of 410 mM and 577 mM lactic acid for fermentations of strain Lp4 and strain Lp4f, respectively. The HPLC analysis of the final fermentation broths (e.g., that of Lp4 and Lp4f) for polyols and organic acids are summarized in Table 7. Fermentation by Lp4 resulted in the production of 410 mM lactic acid, and a total of 6.214 mM of polyols and other organic acids. The fermentation broth for strain Lp4f, yielded a total of 3.139 mM of polyols and other organic acids, and 577 mM of lactic acid. At least partial removal of these fermentation by-products (e.g., polyols and organic acids) and certain unused components of medium could permit obtaining a higher purity (e.g., polymer-grade) lactic acid. The total analyzed impurities produced by these C₂-independant mutants (e.g., Lp4 and Lp4f) during lactic acid fermentation are lower than those produced by fermentation of RWB876 or m850-a.

TABLE 7

| | | Lactic | | | | Malic | Pyruvic | Succinic | Formic | Fumaric |
|---|---|---|---|---|---|---|---|---|---|---|
| | Glucose | acid | Glycerol | Erythritol | Mannitol | acid | acid | acid | acid | acid |
| Strain | (g/L) | (g/L) | (PPM) | (PPM) | (PPM) | (PPM) | (PPM) | (PPM) | (PPM) | (PPM) |
| Lp4 | 19.78 | 36.87 | 0 | 121 | 86 | 64 | 80 | 146 | 95 | 7 |
| Lp4f | 6.44 | 51.90 | 0 | 73 | 0 | 33 | 108 | 123 | 0 | 3 |

### EXAMPLE 7

### Cultivation of Acid-Tolerant Mutant, m850-a, in 10-L Stir Tank for Lactic Acid Production

The fermentation was carried out in a New Brunswick Bioflow 10-L fermenter with a 6-L working volume. The medium composition (M1) was described in Example 1. The aeration was 0.33 vvm with agitation of 250 rpm. The temperature was controlled at 32° C. The pH was not controlled.

After inoculation, the growth phase took place in the first 22-24 hrs in the tank. During the growth phase the ethanol concentration was maintained (by feeding a 25% ethanol solution) to maintain a concentration of between 3-4 g/liter. Once the cell density (OD₆₆₀ₙₘ) reached 10.0 the lactic acid production phase was initiated by adding approximately 70 g/liter glucose and 2.78 g/liter CaCO₃.

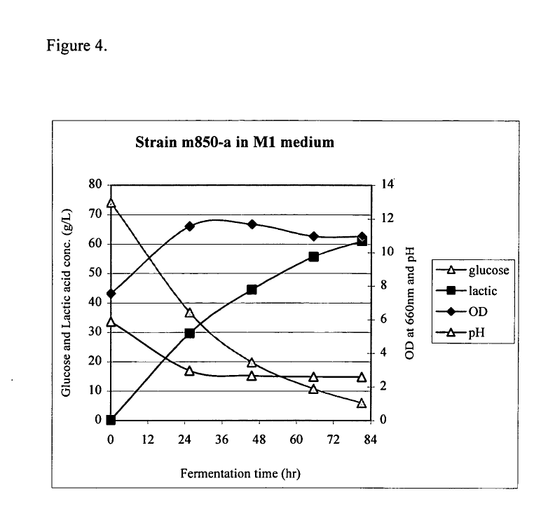

As the fermentation progressed, the pH continued to decrease as shown in FIG. 4. When the glucose concentration reached about 20 g/liter the ethanol feed was decreased, so that the ethanol concentration would be gradually reduced from 3-4 g/liter to 2-3 g/liter, and finally to 1-2 g/liter. At the end of the fermentation both glucose and ethanol were exhausted. Using the conditions described, about 61 g/liter lactic acid was produced from 74 g/liter glucose in 81-hrs with a final pH of 2.60

### References

The following references, to the extent that they provide exemplary procedural or other details supplementary to those set forth herein, are specifically incorporated herein by reference.
Buchta, K. 1983. Lactic Acid, p. 409-417. In Biotechnology, Vol. 3, H. Delleweg (ed.). Verlag Chemie, Weinheim Germany.
Hongo, M., Y. Nomura, and M. Iwahara 1986. Novel methods of lactic acid production by electrodialysis fermentation. Appl. Environ. Microbiol. 32:227-234.
Benninga, H. A. 1990. A history of lactic acid making. Kluwer Academic Publishers, Dordrecht, The Netherlands.
Hohmann, S. 1997. p. 187-211. In Yeast Sugar Metabolism, edited by F. K. Zimmermann et al. and published by Technomic Publishing Company, Inc. Lancaster, Pennsylvania, 17604 U.S.A.
WO 99/19503 Carlson, et al. Low pH lactic acid fermentation (Priority date: 14 Oct. 1997)
Barnett Payne and Yarrow. 1995. Yeasts: characterization and identification, 2^{nd} edition, Cambridge University Press ISBN 052135056.
van Maris, A J. A., M. A. H. Luttik, A. A. Winkler, J. P. van Dijken, and J. T. Pronk. 2003. Overproduction of Threonine Aldolase Circumvents the Biosynthetic Role of Pyruvate Decarboxylase in Glucose-grown Saccharomyces cerevisiae. Appl. Environ. Microbiol. 69:2094-2099.

1. A method of producing lactic acid, comprising:
aerobically culturing in a first culture medium an acid-tolerant (AT) yeast strain that produces essentially no ethanol when cultured in a culture medium,
wherein the AT yeast strain comprises a genome that comprises an exogenous lactate dehydrogenase gene that is capable of being expressed in the AT yeast strain,
wherein a protein resulting from the expression has lactate dehydrogenase activity,
wherein the AT yeast strain is capable of growing in a minimal medium at a lower pH than a parent yeast strain.

2. The method of claim 1, wherein the AT yeast strain is C₂ carbon source independent and is capable of producing lactic acid at a pH of less than about 3.5.

3. The method of claim 1, wherein the AT yeast strain is C₂ carbon source independent and is capable of producing lactic acid at a pH of less than about 2.8.

4. The method of claim 1, wherein the AT yeast strain is C₂ carbon source independent and is capable of producing lactic acid at a pH of less than about 2.3.

5. The method of claim 1, wherein the AT yeast strain is capable of producing greater than about 50 grams lactic acid/100 grams glucose when cultured in the minimal medium comprising glucose as a sole carbon source.

6. The method of claim 1, wherein the AT yeast strain is capable of producing between about 50 and 85 grams lactic acid/100 grams glucose when cultured in the minimal medium comprising glucose as a sole carbon source.

7. The method of claim 1, wherein the AT yeast strain is capable of producing between about 70 and 85 grams lactic acid/100 grams glucose when cultured in the minimal medium comprising glucose as a sole carbon source.

8. The method of claim 1, wherein a culture broth resulting from the culturing of the AT yeast strain comprises less ppm of at least one of glycerol, erythritol, malic acid, pyruvic acid, succinic acid, formic acid, and fumaric acid than a culture broth resulting from the culturing of the parent strain in essentially the same minimal medium under essentially the same culture conditions.

9. The method of claim 1, wherein the AT yeast strain belongs to a genus selected from the group consisting of Saccharomyces, Candida, Schizosaccharomyces, and Kluyveromyces.

10. The method of claim 1, wherein the AT yeast strain is a Saccharomyces cerevisiae.

11. The method of claim 1, wherein the AT yeast strain is a Saccharomyces cerevisiae that has a genotype pdc1(−6,−2)::loxP pdc5(−6,−2)::loxP pdc6(−6,−2)::loxP ura3-52 YEpLpLDH.

12. The method of claim 1, wherein the culturing is performed in an aerobic batch culture, in an aerobic fed-batch culture, or in an aerobic chemostat.

13. The method of claim 1, wherein the AT yeast strain is C₂ carbon source-independent.

14. The method of claim 13, wherein the first culture medium is a minimal medium comprising at least one defined carbon source selected from the group consisting of glucose, sucrose, fructose, maltose, lactose, and galactose.

15. The method of claim 14, wherein glucose is the sole carbon source.

16. The method of claim 1, wherein the AT yeast strain is C₂ carbon source-dependent and the first culture medium is a minimal medium comprising a carbon source consisting essentially of glucose and at least one C₂ carbon source.

17. The method of claim 1, wherein the first culture medium consists essentially of at least one defined carbon source, at least one nitrogen source, monopotassium phosphate, magnesium sulfate, copper sulfate, ferric chloride, manganese sulfate, sodium molybdate, zinc sulphate, biotin, inositol, thiamine, and water, wherein the nitrogen source is selected from the group consisting of urea, ammonium sulfate, ammonium phosphate, and ammonium nitrate.

18. The method of claim 1, wherein a chromosome of the AT yeast strain comprises the exogenous lactate dehydrogenase gene.

19. The method of claim 1, wherein at least one plasmid comprising the exogenous lactate dehydrogenase gene is present in the AT yeast strain.

20. The method of claim 1, wherein the exogenous lactate dehydrogenase gene is a Lactobacillus plantarum, bovine, Lactobacillus casei, Bacillus megaterium, Rhizopus oryzae, or Bacillus stearothermophylus lactate dehydrogenase gene.

21. The method of claim 1, wherein the exogenous lactate dehydrogenase gene is a Lactobacillus plantarum lactate dehydrogenase gene.

22. The method of claim 1, further comprising the step of recovering and purifying the lactic acid or a salt thereof.

23. The method of claim 22, wherein the purification step comprises at least one of distillation, ion exchange, nanofiltration or solvent extraction.

24. An acid-tolerant (AT) yeast strain that produces essentially no ethanol when cultured in a culture medium,
wherein the AT yeast strain comprises a genome that comprises an exogenous lactate dehydrogenase gene that is capable of being expressed in the AT yeast strain,
wherein a protein resulting from the expression has lactate dehydrogenase activity,
wherein the AT yeast strain is capable of producing lactic acid in a minimal medium at a lower pH than a parent yeast strain.

25. The AT yeast strain of claim 24, wherein the AT yeast strain has no detectable amount of pyruvate decarboxylase activity.

26. The AT yeast strain of claim 25, wherein a wild type strain of the AT yeast strain is Crabtree positive.

27. The AT yeast strain of claim 24, wherein the AT yeast strain is capable of producing lactic acid at a pH of less than about 3.5.

28. The AT yeast strain of claim 24, wherein the AT yeast strain is capable of producing lactic acid at a pH of less than about 2.8.

29. The AT yeast strain of claim 24, wherein the AT yeast strain is capable of producing lactic acid at a pH of less than about 2.3.

30. The AT yeast strain of claim 24, wherein the AT yeast strain is capable of producing greater than about 500 mM lactic acid in a culture broth, when cultured aerobically in a minimal medium.

31. The AT yeast strain of claim 30, wherein the AT yeast strain is capable of producing greater than about 565 mM lactic acid.

32. The AT yeast strain of claim 30, wherein the AT yeast strain is capable of producing greater than about 665 mM lactic acid.

33. The AT yeast strain of claim 24, wherein the AT yeast strain belongs to a genus selected from the group consisting of Saccharomyces, Candida, Schizosaccharomyces, Torulaspora, Kluyveromyces, Zygosaccharomyces and Dekkera.

34. The AT yeast strain of claim 24, wherein the AT yeast strain belongs to a genus selected from the group consisting of Saccharomyces, Candida, Schizosaccharomyces, and Kluyveromyces.

35. The AT yeast strain of claim 24, wherein the AT yeast strain belongs to the genus Saccharomyces.

36. The AT yeast strain of claim 24, wherein the AT yeast strain is a Saccharomyces cerevisiae.

37. The AT yeast strain of claim 24, wherein the AT yeast strain is a Saccharomyces cerevisiae that has a genotype pdc1(−6,−2)::loxP pdc5(−6,−2)::loxP pdc6(−6,−2)::loxP ura3-52 YEpLpLDH.

38. The AT yeast strain of claim 24, wherein the AT yeast strain is a Kluyveromyces thermotolerans, a Zygosaccharomyces bailii, a Schizosaccharomyces pombe, or a Candida glabrata.

39. The AT yeast strain of claim 24, wherein the AT yeast strain is C₂ carbon source-independent.

40. The AT yeast strain of claim 39, wherein the AT yeast strain is capable of growing in a minimal medium comprising at least one defined carbon source selected from the group consisting of glucose, sucrose, fructose, maltose, lactose, and galactose.

41. The AT yeast strain of claim 39, wherein the AT yeast strain is capable of growing in a minimal medium comprising glucose as the sole carbon source.

42. The AT yeast strain of claim 24, wherein the AT yeast strain is C₂ carbon source-dependent and the AT yeast strain is capable of growing in a minimal medium comprising a carbon source consisting essentially of glucose and at least one C₂ carbon source.

43. The AT yeast strain of claim 24, wherein the AT yeast strain is capable of growing in a mineral medium consisting essentially of at least one defined carbon source, at least one defined nitrogen source, monopotassium phosphate, magnesium sulfate, copper sulfate, ferric chloride, manganese sulfate, sodium molybdate, zinc sulphate, biotin, inositol, thiamine, and water, wherein the defined nitrogen source is selected from the group consisting of urea, ammonium sulfate, ammonium phosphate, and ammonium nitrate.

44. The AT yeast strain of claim 24, wherein a chromosome of the AT yeast strain comprises the exogenous lactate dehydrogenase gene.

45. The AT yeast strain of claim 24, wherein at least one plasmid comprising the exogenous lactate dehydrogenase gene is present in the AT yeast strain.

46. The AT yeast strain of claim 24, wherein the exogenous lactate dehydrogenase gene is functionally linked to a promoter.

47. The AT yeast strain of claim 46, wherein the promoter is a triose phosphate isomerase promoter.

48. The AT yeast strain of claim 46, wherein the promoter is selected from the group consisting of pyruvate decarboxylase promoters, alcohol dehydrogenase promoters, and L-threonine dehydrogenase promoters.

49. The AT yeast strain of claim 46, wherein the promoter is a Kluyveromyces pyruvate decarboxylase promoter.

50. The AT yeast strain of claim 24, wherein the exogenous lactate dehydrogenase gene is a Lactobacillus. plantarum, bovine, Lactobacillus casei, Bacillus megaterium, Rhizopus oryzae, or Bacillus stearothermophylus lactate dehydrogenase gene.

51. The AT yeast strain of claim 24, wherein the exogenous lactate dehydrogenase gene is a Lactobacillus plantarum lactate dehydrogenase gene.

52. The AT yeast strain of claim 24, wherein the AT yeast strain is capable of producing lactic acid consisting essentially of L-lactic acid.

53. An acid-tolerant (AT) S. cerevisiae that produces essentially no ethanol when cultured in a culture medium,
wherein the AT S. cerevisiae comprises a genome that comprises an exogenous lactate dehydrogenase gene that is capable of being expressed in the AT S. cerevisiae,
wherein a protein resulting from the expression has lactate dehydrogenase activity,
wherein the AT S. cerevisiae is capable of producing lactic acid in a minimal medium at a lower pH than a parent S. cerevisiae strain.

54. The acid-tolerant (AT) S. cerevisiae of claim 53, wherein the AT S. cerevisiae has no detectable amount of pyruvate decarboxylase activity.

55. The acid-tolerant (AT) S. cerevisiae of claim 53, wherein the exogenous lactate dehydrogenase gene is a Lactobacillus plantarum lactate dehydrogenase gene.

56. The acid-tolerant (AT) S. crevisiae of claim 53, wherein at least one plasmid in the AT S. crevisiae comprises the exogenous lactate dehydrogenase gene.

57. The AT S. crevisiae of claim 53, wherein the AT S. crevisiae has a genotype pdc1(−6,−2)::loxP pdc5(−6,−2)::loxP pdc6(−6,−2)::loxP ura3-52 YEpLpLDH.

58. The AT S. crevisiae of claim 53, wherein the AT S. crevisiae is capable of producing greater than about 500 mM lactic acid in a culture broth, when cultured aerobically in a minimal medium.

59. An acid-tolerant C₂ carbon source-independent (CI) yeast strain that produces essentially no ethanol when cultured in a culture medium,
wherein the genome of the yeast strain comprises an exogenous lactate dehydrogenase gene that is capable of being expressed,
wherein a protein resulting from the expression has lactate dehydrogenase activity,
wherein the yeast is capable of producing lactic acid when cultured under aerobic conditions in a first minimal medium comprising glucose as a sole carbon source; and
wherein the CI yeast strain is capable of producing lactic acid in the first minimal medium at a lower pH than a parent strain.

60. The CI yeast strain of claim 59, wherein the CI yeast strain has no detectable amount of pyruvate decarboxylase activity.

61. The CI yeast of strain of claim 60, wherein a wild type yeast of the same strain is Crabtree positive.

62. The CI yeast strain of claim 59, wherein the exogenous lactate dehydrogenase gene is a Lactobacillus plantarum, bovine, Lactobacillus casei, Bacillus megaterium, Rhizopus oryzae, or Bacillus stearothermophylus lactate dehydrogenase gene.

63. The CI yeast strain of claim 59, wherein the exogenous lactate dehydrogenase gene is a Lactobacillus plantarum lactate dehydrogenase gene.

64. The CI yeast strain of claim 59, wherein a chromosome of the CI yeast strain comprises the exogenous lactate dehydrogenase gene.

65. The CI yeast strain of claim 59, wherein at least one plasmid comprising the exogenous lactate dehydrogenase gene is present in the CI yeast strain.

66. The CI yeast strain of claim 65, wherein the at least one plasmid is a 2 micron plasmid.

67. The CI yeast strain of claim 59, wherein the CI yeast strain is capable of producing lactic acid at a pH of less than about 2.8.

68. The CI yeast strain of claim 59, wherein the CI yeast strain is capable of producing lactic acid at a pH of less than about 2.3.

69. The CI yeast strain of claim 59, wherein the CI yeast strain is capable of producing greater than about 50 grams lactic acid/100 grams glucose when cultured in the minimal medium.

70. The CI yeast strain of claim 59, wherein the CI yeast strain is capable of producing between about 50 and 85 grams lactic acid/100 grams glucose when cultured in the minimal medium.

71. The CI yeast strain of claim 59, wherein the CI yeast strain is capable of producing between about 70 and 85 grams lactic acid/100 grams glucose when cultured in the minimal medium.

72. The CI yeast strain of claim 59, wherein the CI yeast strain is capable of producing greater than about 565 mM lactic acid in a culture broth, when cultured aerobically in a second minimal medium.

73. The CI yeast strain of claim 72, wherein the CI yeast strain is capable of producing greater than about 665 mM lactic acid.

74. The CI yeast strain of claim 59, wherein the CI yeast strain belongs to a genus selected from the group consisting of Saccharomyces, Candida, Schizosaccharomyces, Torulaspora, Kluyveromyces, Zygosaccharomyces and Dekkera.

75. The CI yeast strain of claim 59, wherein the CI yeast strain belongs to a genus selected from the group consisting of Saccharomyces, Candida, Schizosaccharomyces, and Kluyveromyces.

76. The CI yeast strain of claim 59, wherein the CI yeast strain is selected from the group consisting of Saccharomyces cerevisiae, Kluyveromyces thermotolerans, Zygosaccharomyces bailii, Schizosaccharomyces pombe, and Candida glabrata

77. The CI yeast strain of claim 59, wherein the CI yeast strain is a Saccharomyces cerevisiae that has a genotype pdc1(−6,−2)::loxP pdc5(−6,−2)::loxP pdc6(−6,−2)::loxP ura3-52 YEpLpLDH.

78. The CI yeast strain of claim 59, wherein the CI yeast strain is capable of growing in a second minimal medium comprising at least one defined carbon source selected from the group consisting of glucose, sucrose, fructose, maltose, lactose, and galactose.

79. The CI yeast strain of claim 59, wherein the CI yeast strain is capable of growing in a second minimal medium consisting essentially of at least one defined carbon source selected from the group consisting of glucose, sucrose, fructose, maltose, lactose, and galactose; at least one nitrogen source selected from the group consisting of urea, ammonium phosphate, ammonium nitrate, and ammonium sulfate; monopotassium phosphate; magnesium sulfate; copper sulfate; ferric chloride; manganese sulfate; sodium molybdate; zinc sulphate; biotin; inositol; thiamine; and water.

80. A minimal culture medium consisting essentially of,
at least one defined carbon source; at least one nitrogen source, monopotassium phosphate, magnesium sulfate, copper sulfate, ferric chloride, manganese sulfate, sodium molybdate, zinc sulphate, biotin, inositol, thiamine, and water.

81. The minimal culture medium of claim 80, wherein the defined carbon source comprises a C₂ carbon source.

82. The minimal culture medium of claim 81, wherein the defined carbon source further comprises at least one compound selected from the group consisting of glucose, sucrose, fructose, lactose, galactose, and maltose.

83. The minimal culture medium of claim 80, wherein glucose is the sole carbon source.

84. The minimal culture medium of claim 80, wherein the minimal culture medium comprises between about 5 g glucose/liter and 100 g glucose/liter.

85. The minimal culture medium of claim 84, wherein the minimal culture medium comprises between 0.1 wt % ethanol and 1 wt % ethanol.

86. The minimal culture medium of claim 80, wherein the minimal culture medium comprises calcium carbonate.

87. The minimal culture medium of claim 80, wherein the nitrogen source comprises at least one compound selected from the group consisting of urea, ammonium sulfate, ammonium nitrate, and ammonium phosphate.

88. The minimal culture medium of claim 80, wherein the minimal culture medium comprises between about 0.5 and 5 g ammonium sulfate/liter.

89. The minimal culture medium of claim 80, wherein the minimal culture medium comprises between about 0.5 and 2 g ammonium sulfate/liter.

90. The minimal culture medium of claim 80, wherein the minimal culture medium comprises between about 1 and 2 g ammonium sulfate/liter.

91. The minimal culture medium of claim 80, wherein the minimal culture medium comprises between about 0.1 and 2 g urea/liter.

92. The minimal culture medium of claim 80, wherein the minimal culture medium comprises between about 0.1 and 1 g urea/liter.

93. The minimal culture medium of claim 80, wherein the minimal culture medium comprises between about 0.5 and 2 g urea/liter.

94. The minimal culture medium of claim 80, wherein the minimal culture medium comprises between about 0.2 and 2 g monopotassium phosphate/liter; between about 0.1 and 1 g magnesium sulfate/liter; between about 5 and 50 micrograms copper sulfate/liter; between about 0.05 and 0.25 mg ferric chloride/liter; between about 0.05 and 0.5 mg manganese sulfate/liter; between about 0.05 and 0.25 mg sodium molybdate/liter; between about 0.05 and 0.5 mg zinc sulphate/liter; between about 0.5 and 2.5 micrograms biotin/liter; between about 0.5 and 4 mg inositol/liter; and between about 0.05 and 0.5 mg thiamine/liter.

95. The minimal culture medium of claim 80, wherein the minimal culture medium comprises between about 5 g glucose/liter and 100 g glucose/liter or between about 0.1 wt % and 1 wt % ethanol, about 5 g ammonium sulfate/liter or about 1 g urea/liter, about 1 g monopotassium phosphate/liter, about 0.5 g magnesium sulfate/liter, about 40 micrograms copper sulfate/liter, about 0.2 mg ferric chloride/liter, about 0.4 mg manganese sulfate/liter, about 0.2 mg sodium molybdate/liter, about 0.4 mg zinc sulphate/liter, about 2 micrograms biotin/liter, about 2 mg inositol/liter, and about 0.4 mg thiamine/liter.

96. A recombinant yeast strain having a genome comprising an exogenous lactate dehydrogenase gene that is capable of being expressed in the recombinant yeast strain,
wherein a protein resulting from the expression has lactate dehydrogenase activity,
wherein the recombinant yeast strain is capable of producing at least about 50 grams lactic acid/100 grams glucose when grown in a minimal medium comprising glucose as the sole carbon source, and
wherein the recombinant yeast strain is capable of growing at a pH of less than about 3.5.

97. The recombinant yeast strain of claim 96, wherein the recombinant yeast strain is capable of producing between about 50 and 85 grams lactic acid/100 grams glucose when grown in the minimal medium.

98. The recombinant yeast strain of claim 96, wherein the recombinant yeast strain is capable of producing between about 70 and 85 grams lactic acid/100 grams glucose when grown in the minimal medium.

99. The recombinant yeast strain of claim 96, wherein the recombinant yeast strain is capable of producing lactic acid at a pH of less than about 2.8.

100. The recombinant yeast strain of claim 96, wherein the recombinant yeast strain is capable of producing lactic acid at a pH of less than about 2.3.

101. The recombinant yeast strain of claim 96, wherein the recombinant yeast strain is capable of producing lactic acid at a pH of less than about 2.0.

102. A method of producing lactic acid, comprising:
aerobically culturing in a first culture medium a recombinant yeast strain having a genome comprising an exogenous lactate dehydrogenase gene that is capable of being expressed in the recombinant yeast strain, wherein a protein resulting from the expression has lactate dehydrogenase activity, wherein the recombinant yeast strain is capable of producing at least about 50 grams lactic acid/100 grams glucose when grown in a minimal medium comprising glucose as the sole carbon source, and wherein the recombinant yeast strain is capable of growing at a pH of less than about 3.5.

103. An acid tolerant (AT) yeast strain that is recovered by a selection process comprising:
(a) growing a first yeast strain in a first aerobic culture, wherein the first aerobic culture is started by inoculating a first minimal medium with a first yeast strain that produces essentially no ethanol when cultured in a culture medium, wherein the first yeast strain comprises a genome that comprises an exogenous lactate dehydrogenase gene that is capable of being expressed in the first yeast strain, wherein a protein resulting from the expression has lactate dehydrogenase activity, wherein during the growth of the first aerobic culture the pH of the culture decreases,
(b) determining about the lowest pH at which the first yeast strain is still capable of growing in the first minimal medium, and
(c) recovering at least one second yeast strain from the first aerobic culture, when the first aerobic culture is still growing, and the pH is about at its lowest.

104. The AT yeast strain of claim 103, wherein the first yeast strain lacks at least one of pyruvate decarboxylase enzyme activity or alcohol dehydrogenase enzyme activity.

105. The AT yeast strain of claim 104, wherein a wild type yeast strain for the first yeast strain is Crabtree positive.

106. The AT yeast strain of claim 103, wherein the selection process further comprises the steps of
(d) growing a second aerobic culture, wherein the second aerobic culture is started by inoculating fresh minimal medium with the recovered second yeast strain wherein during the growth of the second aerobic culture the pH of the culture decreases, and
(e) recovering at least one third yeast strain from the second aerobic culture, when the second aerobic culture is still growing, and the pH is less than about the lowest pH of the first aerobic culture.

107. The AT yeast strain of claim 106, wherein the selection process further comprises repeating steps (d) and (e) at least one time comprising inoculating the fresh minimal medium with a yeast strain recovered from the previous repetition.

108. The AT yeast strain of claim 107, wherein the about lowest pH of the aerobic culture at which the AT yeast strain is growing of the last repetition is less than about the lowest pH of the aerobic culture at which the AT yeast strain is growing of the previous repetition.

109. An acid-tolerant C₂ carbon source-independent (CI) yeast strain that is recovered by a selection process comprising:
(a) inoculating a minimal medium with a first yeast strain having that produces essentially no ethanol when cultured in a culture medium and that requires a C₂ carbon source when glucose is the only other carbon source in the minimal medium , wherein the genome of the yeast strain comprises an exogenous lactate dehydrogenase gene that is capable of being expressed, wherein a protein resulting from the expression has lactate dehydrogenase activity, wherein the yeast is capable of producing lactic acid or salts thereof when cultured under aerobic conditions in a first minimal medium comprising glucose and a C₂ carbon source; and wherein the first yeast strain is capable of growing in the first minimal medium at a lower pH than its parent strain,
(b) culturing the first yeast strain in a series of aerobic batch cultures using a second minimal medium, wherein at the start of the series the second minimal medium comprises glucose and a C₂ carbon source as the sole carbon sources and at concentrations sufficient to permit growth of the yeast culture, wherein the concentration of the C₂ carbon source is decreased over the series of batch cultures, and wherein each successive batch culture is seeded with yeast grown in a batch culture from earlier in the series; and
(c) recovering at least one CI yeast strain from the series of batch cultures that is capable of growing without a C₂ carbon source and with glucose as a sole carbon source.

110. The CI yeast strain of claim 109, wherein the first yeast strain lacks at least one of pyruvate decarboxylase enzyme activity or alcohol dehydrogenase enzyme activity.

111. The CI yeast strain of claim 109, wherein a wild type yeast of the first yeast strain is Crabtree positive.

112. An yeast strain having a deposit number NRRL Y-30696.

113. An yeast strain having a deposit number NRRL Y-30698.

114. A culture medium consisting essentially of water, about 70 g/liter glucose, about 0.5 wt % ethanol, about 1 g/liter urea, about 1 g/liter monopotassium phosphate, about 0.5 g/liter magnesium sulfate heptahydrate, about 2.78 g/liter calcium carbonate, about 62.5 micrograms/liter copper sulfate pentahydrate, about 200 micrograms/liter ferric chloride, about 450 micrograms/liter manganese sulfate monohydrate, about 235 micrograms/liter sodium molybdate dihydrate, about 712 micrograms/liter zinc sulfate heptahydrate, 2 micrograms/liter biotin, 2000 micrograms/liter inositol, and 400 micrograms/liter thiamine hydrochloride.

115. A culture medium comprising between about 400 and 1100 ppm N, between about 215 and 287 ppm K⁺, between about 525 and 700 ppm PO₄⁻², about 49 ppm of Mg⁺², about 195 ppm SO₄⁻², about 1100 ppm of Ca⁺², about 0.07 ppm Fe⁺³, about 0.145 ppm Mn⁺², about 0.09 ppm Mo⁴, about 0.16 ppm Zn⁺², about 0.015 ppm Cu⁺², about 0.002 mg/liter biotin, about 2 mg/liter inositol, and about 0.4 mg/liter thiamine hydrochloride.

116. A fermentation broth comprising:
at least about 500 mM lactic acid and a first group of compounds, wherein the ratio of the mM lactic acid to mM of the first group of compounds in the fermentation is at least about 54, wherein the first group of compounds consists of glycerol, erythritol, mannitol, malic acid, pyruvic acid, succinic acid, formic acid, and fumaric acid.

117. The fermentation broth of claim 116, wherein the pH of the fermentation broth is between about 2.3 and 2.4.

118. The fermentation broth of claim 116, wherein the broth comprises at least about 565 mM lactic acid.

119. The fermentation broth of claim 116, wherein the broth comprises at least about 665 mM lactic acid.

120. The fermentation broth of claim 116, wherein the ratio is greater than about 66.

121. The fermentation broth of claim 116, wherein the ratio is greater than about 184.

122. The fermentation broth of claim 116, wherein the fermentation broth is a product of fermentation by a S. crevisiae strain.

123. A yeast plasmid comprising a yeast replication origin and a Lactobacillus lactate dehydrogenase gene functionally linked to a promoter.

124. The plasmid of claim 122, wherein the replication origin is a 2 micron replication origin.

125. The plasmid of claim 122, wherein the lactate dehydrogenase gene is a L-lactate dehydrogenase gene.

126. The plasmid of claim 122, wherein the lactate dehydrogenase gene is a Lactobacillus plantarum lactate dehydrogenase gene.

127. The plasmid of claim 122, wherein the promoter is a triose phosphate isomerase promoter

128. The plasmid of claim 122, wherein the plasmid is YEpLpLDH.
